SEQUENCE LISTING (35 sequences)

SEQ ID NO: 1 - protein, 5 aa, synthetic construct
GYYMH

SEQ ID NO: 2 - protein, 17 aa, synthetic construct
RINPYTGATTYNQIFKD

SEQ ID NO: 3 - protein, 13 aa, synthetic construct
YYYGNNYNVYLDY

SEQ ID NO: 4 - protein, 11 aa, synthetic construct
SASQGISNYLS

SEQ ID NO: 5 - protein, 7 aa, synthetic construct
YTSTLHS

SEQ ID NO: 6 - protein, 9 aa, synthetic construct
QQYSNLPWT

SEQ ID NO: 7 - protein, 122 aa, synthetic construct
EVQLQQSGPELVKPGASVKISCKASGYSFTGYYMHWVKQSHVKSLEWIGRINPYTGATTYNQIFKDKATLTLDKSSSTAYMELHSLTSEDSAIFYCARYYYGNNYNVYLDYWGQGTTLTVSS

SEQ ID NO: 8 - DNA, 366 bp, synthetic construct
  source 1..366: mol_type = other DNA
gaggttcagctgcagcagtctggccctgagcttgtgaaacctggcgcctccgtgaagatcagctgtaaagccagcggctacagcttcaccggctactacatgcactgggtcaagcagtcccacgtgaagtccctggaatggatcggccggatcaacccttacaccggcgccaccacctacaaccagatcttcaaggacaaggccacactgaccctggacaagagcagcagcaccgcctacatggaactgcacagcctgaccagcgaggacagcgccatcttctactgtgcccggtactactacggcaacaactacaacgtgtacctggactactggggccagggcacaaccctgacagtttcttct

SEQ ID NO: 9 - protein, 107 aa, synthetic construct
DIQLTQTTSSLSASLGDRVTISCSASQGISNYLSWYQQKPDGTFKLLIYYTSTLHSGVPSRFSGSGSGTDYSLTISNLEPEDIATYYCQQYSNLPWTFGGGTRLEIK

SEQ ID NO: 10 - DNA, 321 bp, synthetic construct
  source 1..321: mol_type = other DNA
gatattcagctgacccagaccaccagcagcctgtctgcctctctgggcgatagagtgaccatcagctgttctgccagccagggcatcagcaactacctgagctggtatcagcagaagcccgacggcaccttcaagctgctgatctactacaccagcacactgcactccggcgtgcccagcagattttctggaagcggaagcggcaccgactactccctgaccatctccaacctggaacctgaggatatcgccacctactactgccagcagtacagcaacctgccttggacctttggcggcggaacccggctggaaatcaaa

SEQ ID NO: 11 - protein, 15 aa, synthetic construct
GGGGSGGGGSGGGGS

SEQ ID NO: 12 - protein, 244 aa, synthetic construct
EVQLQQSGPELVKPGASVKISCKASGYSFTGYYMHWVKQSHVKSLEWIGRINPYTGATTYNQIFKDKATLTLDKSSSTAYMELHSLTSEDSAIFYCARYYYGNNYNVYLDYWGQGTTLTVSSGGGGSGGGGSGGGGSDIQLTQTTSSLSASLGDRVTISCSASQGISNYLSWYQQKPDGTFKLLIYYTSTLHSGVPSRFSGSGSGTDYSLTISNLEPEDIATYYCQQYSNLPWTFGGGTRLEIK

SEQ ID NO: 13 - DNA, 732 bp, synthetic construct
  source 1..732: mol_type = other DNA
gaggttcagctgcagcagtctggccctgagcttgtgaaacctggcgcctccgtgaagatcagctgtaaagccagcggctacagcttcaccggctactacatgcactgggtcaagcagtcccacgtgaagtccctggaatggatcggccggatcaacccttacaccggcgccaccacctacaaccagatcttcaaggacaaggccacactgaccctggacaagagcagcagcaccgcctacatggaactgcacagcctgaccagcgaggacagcgccatcttctactgtgcccggtactactacggcaacaactacaacgtgtacctggactactggggccagggcacaaccctgacagtttcttctggtggcggaggatctggcggaggtggaagcggcggaggcggatctgatattcagctgacccagaccaccagcagcctgtctgcctctctgggcgatagagtgaccatcagctgttctgccagccagggcatcagcaactacctgagctggtatcagcagaagcccgacggcaccttcaagctgctgatctactacaccagcacactgcactccggcgtgcccagcagattttctggaagcggaagcggcaccgactactccctgaccatctccaacctggaacctgaggatatcgccacctactactgccagcagtacagcaacctgccttggacctttggcggcggaacccggctggaaatcaaa

SEQ ID NO: 14 - protein, 43 aa, synthetic construct
GAAAIEVMYPPPYLDNEKSNGTIIHVKGKHLCPSPLFPGPSKP

SEQ ID NO: 15 - DNA, 129 bp, synthetic construct
  source 1..129: mol_type = other DNA
ggcgccgctgctattgaagtgatgtaccctcctccttacctggacaacgagaagtccaacggcaccatcatccacgtgaagggcaagcacctgtgtccttctccactgttccccggacctagcaagcct

SEQ ID NO: 16 - protein, 45 aa, synthetic construct
TTTPAPRPPTPAPTIASQPLSLRPEACRPAAGGAVHTRGLDFACD

SEQ ID NO: 17 - DNA, 135 bp, synthetic construct
  source 1..135: mol_type = other DNA
accaccacaccagctcctcggccacctactccagctccaacaattgccagccagcctctgtctctgaggcccgaagcttgtagacctgctgctggcggagccgtgcatacaagaggactggatttcgcctgcgac

SEQ ID NO: 18 - protein, 27 aa, synthetic construct
FWVLVVVGGVLACYSLLVTVAFIIFWV

SEQ ID NO: 19 - DNA, 81 bp, synthetic construct
  source 1..81: mol_type = other DNA
ttctgggtgctcgttgttgttggcggcgtgctggcctgttattccctgctggttaccgtggccttcatcatcttttgggtc

SEQ ID NO: 20 - protein, 24 aa, synthetic construct
IYIWAPLAGTCGVLLLSLVITLYC

SEQ ID NO: 21 - DNA, 72 bp, synthetic construct
  source 1..72: mol_type = other DNA
atctacatctgggcccctctggctggaacatgtggcgtgctgctgctcagcctggtcatcaccctgtactgc

SEQ ID NO: 22 - protein, 40 aa, synthetic construct
RSKRSRLLHSDYMNMTPRRPGPTRKHYQPYAPPRDFAAYR

SEQ ID NO: 23 - DNA, 120 bp, synthetic construct
  source 1..120: mol_type = other DNA
cgaagcaagcggagccggctgctgcacagcgactacatgaacatgacccctagacggcccggaccaaccagaaagcactaccagccttacgctcctcctagagacttcgccgcctaccgg

SEQ ID NO: 24 - protein, 42 aa, synthetic construct
KRGRKKLLYIFKQPFMRPVQTTQEEDGCSCRFPEEEEGGCEL

SEQ ID NO: 25 - DNA, 126 bp, synthetic construct
  source 1..126: mol_type = other DNA
aagcggggcagaaagaagctgctgtacatcttcaagcagcccttcatgcggcccgtgcagaccacacaagaggaagatggctgctcctgcagattccccgaggaagaagaaggcggctgcgagctg

SEQ ID NO: 26 - protein, 111 aa, synthetic construct
VKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 333 bp, synthetic construct
  source 1..333: mol_type = other DNA
gtgaagttcagcagaagcgccgacgctcctgcctatcagcagggacagaatcagctgtacaacgagctgaacctggggcgcagagaagagtacgacgtgctggacaagagaagaggcagggaccctgagatgggcggcaagcccagaagaaagaaccctcaagagggcctgtataatgagctgcagaaagacaagatggccgaggcctacagcgagatcggaatgaagggcgaacgcagaagaggaaagggccacgacggactgtatcagggcctgagcacagccaccaaggacacctatgatgccctgcacatgcaggcactgcctccaaga

SEQ ID NO: 28 - protein, 21 aa, synthetic construct
MALPVTALLLPLALLLHAARP

SEQ ID NO: 29 - DNA, 63 bp, synthetic construct
  source 1..63: mol_type = other DNA
atggctctgcctgttacagctctgctgctgcctctggctctgcttctgcatgccgctagacct

SEQ ID NO: 30 - protein, 26 aa, synthetic construct
MVATLLVTSLLLCELPHPAFLLIPDT

SEQ ID NO: 31 - DNA, 78 bp, synthetic construct
  source 1..78: mol_type = other DNA
atggttgctacactgctggtcacaagcctgctgctgtgcgagcttccccatcctgcctttctgctgatccccgataca

SEQ ID NO: 32 - protein, 487 aa, synthetic construct
MALPVTALLLPLALLLHAARPEVQLQQSGPELVKPGASVKISCKASGYSFTGYYMHWVKQSHVKSLEWIGRINPYTGATTYNQIFKDKATLTLDKSSSTAYMELHSLTSEDSAIFYCARYYYGNNYNVYLDYWGQGTTLTVSSGGGGSGGGGSGGGGSDIQLTQTTSSLSASLGDRVTISCSASQGISNYLSWYQQKPDGTFKLLIYYTSTLHSGVPSRFSGSGSGTDYSLTISNLEPEDIATYYCQQYSNLPWTFGGGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - DNA, 1461 bp, synthetic construct
  source 1..1461: mol_type = other DNA
atggctctgcctgttacagctctgctgctgcctctggctctgcttctgcatgccgctagacctgaggttcagctgcagcagtctggccctgagcttgtgaaacctggcgcctccgtgaagatcagctgtaaagccagcggctacagcttcaccggctactacatgcactgggtcaagcagtcccacgtgaagtccctggaatggatcggccggatcaacccttacaccggcgccaccacctacaaccagatcttcaaggacaaggccacactgaccctggacaagagcagcagcaccgcctacatggaactgcacagcctgaccagcgaggacagcgccatcttctactgtgcccggtactactacggcaacaactacaacgtgtacctggactactggggccagggcacaaccctgacagtttcttctggtggcggaggatctggcggaggtggaagcggcggaggcggatctgatattcagctgacccagaccaccagcagcctgtctgcctctctgggcgatagagtgaccatcagctgttctgccagccagggcatcagcaactacctgagctggtatcagcagaagcccgacggcaccttcaagctgctgatctactacaccagcacactgcactccggcgtgcccagcagattttctggaagcggaagcggcaccgactactccctgaccatctccaacctggaacctgaggatatcgccacctactactgccagcagtacagcaacctgccttggacctttggcggcggaacccggctggaaatcaaaaccaccacaccagctcctcggccacctactccagctccaacaattgccagccagcctctgtctctgaggcccgaagcttgtagacctgctgctggcggagccgtgcatacaagaggactggatttcgcctgcgacatctacatctgggcccctctggctggaacatgtggcgtgctgctgctcagcctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctggtgaagttcagcagaagcgccgacgctcctgcctatcagcagggacagaatcagctgtacaacgagctgaacctggggcgcagagaagagtacgacgtgctggacaagagaagaggcagggaccctgagatgggcggcaagcccagaagaaagaaccctcaagagggcctgtataatgagctgcagaaagacaagatggccgaggcctacagcgagatcggaatgaagggcgaacgcagaagaggaaagggccacgacggactgtatcagggcctgagcacagccaccaaggacacctatgatgccctgcacatgcaggcactgcctccaaga

SEQ ID NO: 34 - protein, 486 aa, synthetic construct
MALPVTALLLPLALLLHAARPEVQLQQSGPELVKPGASVKISCKASGYSFTGYYMHWVKQSHVKSLEWIGRINPYTGATTYNQIFKDKATLTLDKSSSTAYMELHSLTSEDSAIFYCARYYYGNNYNVYLDYWGQGTTLTVSSGGGGSGGGGSGGGGSDIQLTQTTSSLSASLGDRVTISCSASQGISNYLSWYQQKPDGTFKLLIYYTSTLHSGVPSRFSGSGSGTDYSLTISNLEPEDIATYYCQQYSNLPWTFGGGTRLEIKGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - DNA, 1458 bp, synthetic construct
  source 1..1458: mol_type = other DNA
atggctctgcctgttacagctctgctgctgcctctggctctgcttctgcatgccgctagacctgaggttcagctgcagcagtctggccctgagcttgtgaaacctggcgcctccgtgaagatcagctgtaaagccagcggctacagcttcaccggctactacatgcactgggtcaagcagtcccacgtgaagtccctggaatggatcggccggatcaacccttacaccggcgccaccacctacaaccagatcttcaaggacaaggccacactgaccctggacaagagcagcagcaccgcctacatggaactgcacagcctgaccagcgaggacagcgccatcttctactgtgcccggtactactacggcaacaactacaacgtgtacctggactactggggccagggcacaaccctgacagtttcttctggtggcggaggatctggcggaggtggaagcggcggaggcggatctgatattcagctgacccagaccaccagcagcctgtctgcctctctgggcgatagagtgaccatcagctgttctgccagccagggcatcagcaactacctgagctggtatcagcagaagcccgacggcaccttcaagctgctgatctactacaccagcacactgcactccggcgtgcccagcagattttctggaagcggaagcggcaccgactactccctgaccatctccaacctggaacctgaggatatcgccacctactactgccagcagtacagcaacctgccttggacctttggcggcggaacccggctggaaatcaaaggcgccgctgctattgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttattccctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctaccgggtgaagttcagcagaagcgccgacgctcctgcctatcagcagggacagaatcagctgtacaacgagctgaacctggggcgcagagaagagtacgacgtgctggacaagagaagaggcagggaccctgagatgggcggcaagcccagaagaaagaaccctcaagagggcctgtataatgagctgcagaaagacaagatggccgaggcctacagcgagatcggaatgaagggcgaacgcagaagaggaaagggccacgacggactgtatcagggcctgagcacagccaccaaggacacctatgatgccctgcacatgcaggcactgcctccaaga

(19) United States
(12) Patent Application Publication
(10) Pub. No.: US 2025/0032616 A1
(43) Pub. Date: Jan. 30, 2025

(54) ANTI-CD99 CAR-T CELLS FOR THE TREATMENT OF CANCER

(71) Applicant: The Regents of the University of Colorado, A Body Corporate, Denver, CO (US)

(72) Inventors: Sujatha VENKATARAMAN, Aurora, CO (US); Rajeev VIBHAKAR, Denver, CO (US)

(21) Appl. No.: 18/601,461

(22) Filed: Mar. 11, 2024

Related U.S. Application Data
(60) Provisional application No. 63/489,718, filed on Mar. 10, 2023.

(51) Int. Cl. A61K 39/00; A61P 35/00; C07K 14/725; C07K 16/28

(52) U.S. Cl. CPC A61K 39/464429; A61K 39/4611; A61K 39/4631; A61P 35/00; C07K 14/7051; C07K 16/2896; C07K 2317/622

(57) ABSTRACT

The present disclosure provides compositions and methods directed to anti-CD99 CAR-T therapies for treating cancer, such as DIPG. Ewing Sarcoma, and AML.

### RELATED APPLICATIONS

This application claims priority to, and the benefit of, U.S. Provisional Application No. 63/489,718, filed on Mar. 10, 2023, the contents of which is incorporated herein by reference in its entirety.

### SEQUENCE LISTING

The Sequence Listing XML associated with this application is provided electronically in XML file format and is hereby incorporated by reference into the specification. The name of the XML file containing the Sequence Listing XML is “UNCO-065_001US_SeqList”. The XML file is 38,292 bytes, created on Mar. 11, 2024, and is being submitted electronically via USPTO Patent Center.

### BACKGROUND

Diffuse intrinsic pontine glioma (DIPGs) is the most aggressive pediatric brain tumor and the leading cause of brain tumor-related death in children. DIPG diffusely involves the pons, making it inoperable and resulting in a median survival of 11 months for children with this tumor. Radiation therapy provides only temporary relief and chemotherapy is not effective. For the same reasons, the 5-year survival rate has held steady at 0% since 1950. Recent studies have shown that a somatic Lys27Met substitution in histone 3.3 (H3.3K27M mutation) occurs in more than 85% of patients with DIPG and is associated with poor survival. While these studies give crucial insight into driver mutations they have not yet resulted in new therapeutic options. Thus, there is a critical need to identify and validate more effective, biologically based therapies to target DIPG, including DIPG that specifically arises from the presence of H3K27M mutation.

Recently, Chimeric Antigen Receptor (CAR) T cells targeting DIPG have shown promise in preclinical models and are in development for clinical trials. However, while some CAR-T cells initially demonstrate complete clearance of tumor burden in DIPG xenograft models, tumor recurrence often occurs. Without wishing to be limited by theory, recurrences likely due to loss of the CAR-T cells as they also express the target antigen, CD99 (i.e. cellular fratricide). Accordingly, there is a need in the art for improved CAR-T cell treatments for DIPG.

In addition to DIPG, CAR polypeptides and CAR-T cells of the present disclosure, can also be used in the treatment of other cancers such as acute myeloid leukemia (AML) and Ewing sarcoma.

AML is a blood cancer in which the bone marrow of a subject makes abnormal myeloblasts, red blood cells, or platelets. AML is one of the most common forms of acute leukemia in adults. The build-up of AML cells in bone marrow and blood can rapidly lead to infection, anemia, excessive bleeding and death. BCL-2 inhibitor venetoclax has recently emerged as an important component of therapy for acute myeloid leukemia (AML). The current FDA-approved standard of care for the majority of patients who are too elderly or unfit for aggressive chemotherapy is treatment with venetoclax in combination with a hypomethylating agent, such as azacitidine (“Ven/aza treatment”) or decitabine. It is estimated that approximately 70% of these patients will achieve complete remission (CR) of their disease upon Ven/aza treatment. However, it is estimated that approximately 30% of patients do not respond to treatment with ven/aza and are unable to achieve CR. Accordingly, there is an unmet need in the art for improved compositions and methods for treating AML, including in subjects that will not respond to treatment with Ven/aza.

Ewing sarcoma is a type of cancer that occurs primarily in the bone or soft tissue. While Ewing sarcoma can develop in any bone, it is most often found in the hip bones, ribs, or long bones (e.g., femur, tibia or humerus). It can involve the muscle and the soft tissues around the tumor as well. Ewing sarcoma cells can also metastasize (spread) to other areas of the body, including the bone marrow, lungs, kidneys, heart, adrenal glands and other soft tissues. As the second-most common type of bone cancer affecting children and young adults, it accounts for about 1 percent of childhood cancers. About 225 children and adolescents are diagnosed with Ewing sarcoma in the U.S. each year. While Ewing sarcoma can occur at any time during childhood, it most commonly develops during puberty, when bones are growing rapidly. Ewing sarcoma most often occurs in children between the ages of 10 and 20. Over the last 40 years, both local therapy and multiagent adjuvant chemotherapy have achieved considerable progress in the treatment of localized disease that improved the 5-year survival rate from less than 20% to greater than 70%, but the recurrence rate remains high. However, most present locally, and subclinical metastatic disease is present in almost all cases. Approximately 25% of patients with initially localized disease ultimately relapse. No standard therapy exists for relapsed and refractory Ewing sarcoma, with survival rates being less than 30% in those with isolated lung metastases and less than 20% in those with bone and bone marrow involvement. Accordingly, there is an unmet need in the art for improved compositions and methods for treating Ewing sarcoma, including in subjects with relapsed and refractory disease.

### SUMMARY

The present disclosure provides anti-CD99 single chain variable fragments (scFvs) comprising the amino acid sequence of SEQ ID NO: 12.

The present disclosure provides chimeric antigen receptor (CAR) polypeptides comprising the anti-CD99 scFvs of the present disclosure.

In some aspects, a CAR polypeptide comprises, from N-terminus to C-terminus: i) a signal peptide; ii) an anti-CD99 antigen binding domain comprising the anti-CD99 scFv of claim 1; iii) a hinge domain comprising a CD8 hinge polypeptide; iv) a transmembrane domain comprising a CD8 transmembrane polypeptide; v) a costimulatory domain comprising a 4-1BB costimulatory polypeptide; and vi) an activation domain comprising CD3ζ activation polypeptide. In some aspects, the CD8 hinge polypeptide comprises the amino acid sequence of SEQ ID NO: 16; the CD8 transmembrane polypeptide comprises the amino acid sequence of SEQ ID NO: 20; the 4-1BB costimulatory polypeptide comprises the amino acid sequence of SEQ ID NO: 24; and the CD3ζ activation polypeptide comprises the amino acid sequence of SEQ ID NO: 26. In some aspects, a CAR polypeptide comprises the amino acid sequence of SEQ ID NO: 32.

In some aspects, a CAR polypeptide comprises, from N-terminus to C-terminus: i) a signal peptide; ii) an anti-CD99 antigen binding domain comprising the anti-CD99 scFv of claim 1; iii) a hinge domain comprising a CD28 hinge polypeptide; iv) a transmembrane domain comprising a CD28 transmembrane polypeptide; v) a costimulatory domain comprising a CD28 costimulatory polypeptide; and vi) an activation domain comprising CD3ζ activation polypeptide. In some aspects, the CD28 hinge polypeptide comprises the amino acid sequence of SEQ ID NO: 14; the CD28 transmembrane polypeptide comprises the amino acid sequence of SEQ ID NO: 18; the CD28 costimulatory polypeptide comprises the amino acid sequence of SEQ ID NO: 22; and the CD3ζ activation polypeptide comprises the amino acid sequence of SEQ ID NO: 26. In some aspects, a CAR polypeptide comprises the amino acid sequence of SEQ ID NO: 34.

The present disclosure provides nucleic acid molecules comprising at least one nucleic acid sequence encoding for the anti-CD99 scFvs of the present disclosure or the CAR polypeptides of the present disclosure.

The present disclosure provides vectors comprising the nucleic acid molecules of the present disclosure. In some aspects, a vector is a viral vector. In some aspects, a viral vector is an AAV vector or a lentiviral vector.

The present disclosure provides cells expressing the CAR polypeptide of the present disclosure. In some aspects, the cell is an immune cell. In some aspects, the immune cell is a T cell, NK cell, NK-like cell, NKT cell, or cytokine induced killer (CIK) cell, preferably wherein the immune cell is a T cell.

In some aspects, the cells of the present disclosure do not express CD99. In some aspects, the cells have been genetically modified to not express CD99. In some aspects, the genetic modification was performed using a CRISPR-based genetic modification system.

The present disclosure provides populations of the cells of the present disclosure.

The present disclosure provides methods of treating cancer in a subject, the methods comprising administering to the subject one or more amounts of the cell population of the present disclosure.

The present disclosure provides methods of preventing cancer metastasis in a subject, the method comprising administering to the subject one or more amounts of the cell population of the present disclosure.

In some aspects, the methods further comprise administering to the subject at least one additional therapy. In some aspects, the at least one additional therapy comprises at least one of radiation therapy, chemotherapy, and surgery.

In some aspects, the cancer is diffuse intrinsic pontine glioma (DIPG), acute myeloid leukemia (AML) or Ewing sarcoma.

Any of the above aspects, or any of the aspects described herein, can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The above and further features will be more clearly appreciated from the following detailed description when taken in conjunction with the accompanying drawings.

FIG. 1 shows ELISA measurements of cytokine production in normal human astrocyte cells, H3K27M+ tumor cells, or H3K27M-KO tumor cells treated with CD99(10D1)-BBz CAR T cells. NHA; normal human astrocytes.

FIG. 2 shows XCELLigence real-time cell proliferation measurements when BT245, NHA, and UPN-1525 cells were co-incubated with anti-CD19 or CD99(10D1)-BBz CAR-T cells. NHA; normal human astrocytes; UPN-1525; primary H3K27M-DIPG patient tumor cells.

FIGS. 3A-3B shows the functionality of CD99(10D1)-BBz CAR-T cells in DIPG tumor cells. FIG. 3A shows XCELLigence real-time cell proliferation measurements of DIPG tumor cells treated with CD99(10D1)-BBz CAR-T cells at a 1:1 (tumor cell: CAR-T cell) ratio compared to untreated DIPG4 cells and DIPG4 cells treated with anti-CD19 CAR-T cells. FIG. 3B shows ELISA measurements of cytokine production when DIPG4 cells were treated with CD99(10D1)-BBz CAR-T cells at a 1:1 ratio compared to DIPG4 cells treated with anti-CD19 CAR-T cells.

FIGS. 4A-4B shows the functionality of CD99(10D1)-BBz CAR-T cells in primary patient-derived DIPG tumor cells. FIG. 4A shows XCELLigence real-time cell proliferation measurements of primary patient-derived DIPG tumor cells (MAF-002) treated with CD99(10D1)-BBz CAR-T cells at a 1:1 (tumor cell: CAR-T cell) ratio compared to untreated MAF-002 cells and MAF-002 cells treated with anti-CD19 CAR-T cells. FIG. 4B shows ELISA measurements of cytokine production when MAF-002 cells were treated with CD99(10D1)-BBz CAR-T cells at a 1:1 ratio compared to MAF-002 cells treated with anti-CD19 CAR-T cells.

FIGS. 5A-5B show the efficacy of CD99(10D1)-BBz CAR-T treatment against DIPG tumors. FIG. 5A shows Kaplan Meier's survival analysis of the BT245-Luciferase pons-bearing DIPG tumor mouse model post single-dose infusion of CD99(10D1)-BBz CAR-T cells compared to anti-CD19 CAR-T cells. FIG. 5B shows Kaplan Meier's survival analysis of the DIPG13-Luciferase (DIPGXIII*) pons-bearing DIPG tumor mouse model post single dose infusion of CD99(10D1)-BBz CAR-T cells compared to anti-CD19 CAR-T cells.

FIG. 6 shows Kaplan Meier survival analysis of BT245-DIPG-Luciferase mouse model after loco-regional delivery of CD99(10D1)-BBz CAR-T cells compared to anti-CD19 CAR-T cells (Mock CAR-T).

FIG. 7 shows Kaplan Meier survival analysis of DIPG13-Luciferase mouse model after loco-regional delivery of CD99(10D1)-BBz CAR-T cells compared to anti-CD19 CAR-T cells (Mock CAR-T).

FIG. 8 shows a schematic of the protocol for generating CRISPR/Cas 9 edited CD99KO CD99(10D1)-BBz CAR-T cells.

FIGS. 9A-9B shows the functionality of unedited CD99(10D1)-BBz CAR-T cells and edited CD99KO CD99(10D1)-BBz CAR-T cells in DIPG tumor cells. FIG. 9A shows XCELLigence real-time cell proliferation measurements of DIPG4 tumor cells treated with unedited CD99(10D1)-BBz CAR-T cells and edited CD99KO CD99(10D1)-BBz CAR-T cells ((CD99KO) CAR-T99) compared to DIPG4 tumor cells treated with anti-CD19 CAR-T cells (Control). FIG. 9B shows ELISA measurements of cytokine production when DIPG4 tumor cells were treated with unedited CD99(10D1)-BBz CAR-T cells or edited CD99KO CD99(10D1)-BBz CAR-T cells at a 1:1 ratio.

FIGS. 10A-10B show the survival of DIPG tumor bearing mouse models treated with unedited CD99(10D1) CAR-T cells and edited CD99KO CD99(10D1)-BBz CAR-T cells. FIG. 10A shows Kaplan Meier survival analysis of BT245-GFP-Luciferase DIPG tumor bearing mouse model after unedited CD99(10D1)-BBz CAR-T cell and edited CD99KO CD99(10D1)-BBz CAR-T cell treatment, compared to anti-CD19 CAR-T cell treatment. FIG. 10B shows Kaplan Meier survival analysis of DIPG13-GFP-Luciferase DIPG tumor bearing mouse model after unedited CD99(10D1)-BBz CAR-T cell and edited CD99KO CD99(10D1)-BBz CAR-T cell treatment, compared to anti-CD19 CAR-T cell treatment.

FIG. 11 show the efficacy of CD99(10D1)-BBz CAR-T cells in Ewing Sarcoma. FIG. 11 shows XCELLigence real-time cell proliferation measurements of different Ewing Sarcoma human donor cells treated with CD99(10D1)-BBz CAR-T cells as compared to treatment with anti-CD19 CAR-T cells (Mock CAR).

FIGS. 12A-12B shows the functionality of CD99(10D1)-BBz CAR-T cells in Ewing Sarcoma cell lines. FIG. 12A shows XCELLigence real-time cell proliferation measurements of Ewing Sarcoma cell lines (A673 and TC71) treated with CD99(10D1)-BBz CAR-T cells at a 1:1 (tumor cell: CAR-T cell) ratio compared to Ewing Sarcoma cells treated with anti-CD19 CAR-T cells. FIG. 12B shows ELISA measurements of cytokine production when Ewing Sarcoma cells were treated with CD99(10D1)-BBz CAR-T cells at a 1:1 ratio compared to Ewing Sarcoma cells treated with anti-CD19 CAR-T cells.

FIGS. 13A-13B show the in vivo efficacy of CD99(10D1)-BBz CAR-T treatment in Ewing Sarcoma mouse model. FIG. 13A shows prolonged xenograft survival after limb amputation in CD99(10D1)-BBz CAR-T cell treated mice as compared to mice treated with anti-CD19 CAR-T cells. FIG. 13B shows lung metastasis, as measured by the increase in detection of EQS-FLI1 DNA in the plasma of treated mice, in anti-CD19 CAR-T cell treated mice as compared to CD99(10D1)-BBz CAR-T cell treated mice.

FIGS. 14A-14B shows the functionality of unedited CD99(10D1)-BBz CAR-T cells and CRISPR/Cas9 edited CD99KO CD99(10D1)-BBz CAR-T cells in Ewing Sarcoma cell lines.

FIG. 14A shows XCELLigence real-time cell proliferation measurements of an Ewing Sarcoma cell line (TC71) treated with unedited CD99(10D1)-BBz CAR-T cells or edited CD99KO CD99(10D1)-BBz CAR-T cells at a 1:1 (tumor cell: CAR-T cell) ratio compared to Ewing Sarcoma cells treated with anti-CD19 CAR-T cells. FIG. 14B shows ELISA measurements of cytokine production when Ewing Sarcoma cells were treated with unedited CD99(10D1)-BBz CAR-T cells or edited CD99KO CD99(10D1)-BBz CAR-T cells at a 1:1 ratio compared to Ewing Sarcoma cells treated with anti-CD19 CAR-T cells.

FIG. 15 shows prolonged xenograft survival in edited CD99KO CD99(10D1)-BBz CAR-T cell treated mice as compared to mice treated with anti-CD19 CAR-T cells or unedited CD99(10D1)-BBz CAR-T cells.

FIG. 16 shows flow cytometry plots showing the presence of unedited CD99KO CD99(10D1)-BBz CAR-T cells persisting in more than 25% of human CAR+ cells in mice infused with edited CD99KO CD99(10D1)-BBz CAR-T cells, as compared to the undetected anti-CD19 CAR-T cells and unedited CD99(10D1)-BBz CAR-T cells.

FIG. 17 shows a schematic representation of the in vivo results obtained by treating primary Ewing Sarcoma tumors implanted in the mouse pretibial space.

### DETAILED DESCRIPTION

Anti-CD99 scFv

The present disclosure provides anti-CD99 single chain variable fragments (scFv).

As would be appreciated by the skilled artisan, a single chain variable fragments (scFv) are a monovalent molecule comprising the two domains of the Fv fragment (i.e., VL and VH) of an antibody which are joined by a synthetic linker, which enables the two domains to be synthesized as a single polypeptide chain (see, e.g., Bird et al. (1988), Science 242:423-6; Huston et al. (1988) Proc. Natl. Acad. Sci. USA 85:5879-83; and Osbourn et al. (1998) Nat. Biotechnol. 16:778-81).

“Antibody” as used herein refers to monoclonal or polyclonal antibodies. The term “monoclonal antibodies,” as used herein, refers to antibodies that are produced by a single clone of B-cells and bind to the same epitope. In contrast, “polyclonal antibodies” refer to a population of antibodies that are produced by different B-cells and bind to different epitopes of the same antigen. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CHL CH2 and CH3) regions, and each light chain contains one N-terminal variable (VL) region and one C-terminal constant (CL) region. The variable regions of each pair of light and heavy chains form the antigen binding site of an antibody. The VH and VL regions have a similar general structure, with each region comprising four framework regions, whose sequences are relatively conserved. The framework regions are connected by three complementarity determining regions (CDRs). The three CDRs, known as CDR1, CDR2, and CDR3, form the “hypervariable region” of an antibody, which is responsible for antigen binding. As would be appreciated by the skilled artisan, the three CDRs of the VH region can be referred to as CDRH1, CDRH2, and CDRH3 and the three CDRs of the VL region can be referred to as CDRL1, CDRL2, CDRL3.

In some aspects, the CDRs are the Kabat CDRs. In other aspects, the CDRs are the Chothia CDRs. In other aspects, the CDRs are IMGT CDRs. In other words, in aspects with more than one CDR, the CDRs may be any of Kabat, Chothia, IMGT combination CDRs, or combinations thereof.

In some aspects, an anti-CD99 scFv can comprise a CDHR1 having an amino acid sequence that is at least 97%, 98%, or 99% identical to SEQ ID NO: 1, a CDRH2 having an amino acid sequence that is at least 97%, 98%, or 99% identical to SEQ ID NO: 2, a CDRH3 having an amino acid sequence that is at least 97%, 98%, or 99% identical to SEQ ID NO: 3, a CDRL1 having an amino acid sequence that is at least 97%, 98%, or 99% identical to SEQ ID NO: 4, a CDRL2 having an amino acid sequence that is at least 97%, 98%, or 99% identical to SEQ ID NO: 5, a CDHL3 having an amino acid sequence that is at least 97%, 98%, or 99% identical to SEQ ID NO: 6.

In some aspects, an anti-CD99 scFv can comprise a CDHR1 having an amino acid sequence of SEQ ID NO: 1, a CDRH2 having an amino acid sequence of SEQ ID NO: 2, a CDRH3 having an amino acid sequence of SEQ ID NO: 3, a CDRL1 having an amino acid sequence of SEQ ID NO: 4, a CDRL2 having an amino acid sequence of SEQ ID NO: 5, a CDHL3 having an amino acid sequence of SEQ ID NO: 6.

In some aspects, an anti-CD99 scFv can comprise a VH domain comprising an amino acid sequence that is at least 97%, 98%, or 99% identical to SEQ ID NO: 7. Accordingly, a nucleic acid sequence encoding for VH domain can comprise, consist essentially of, or consist of a nucleic acid sequence that is at least 65%, 70%, 75%, 80%, 85%, 90%, 95%, 96%, 97%, 98%, 99% or 100% (or any percentage in between) identical to SEQ ID NO: 8.

In some aspects, an anti-CD99 scFV can comprise a VL domain comprising an amino acid sequence that is at least 97%, 98%, or 99% identical to SEQ ID NO: 9. Accordingly, a nucleic acid sequence encoding for VL domain can comprise, consist essentially of, or consist of a nucleic acid sequence that is at least 65%, 70%, 75%, 80%, 85%, 90%, 95%, 96%, 97%, 98%, 99% or 100% (or any percentage in between) identical to SEQ ID NO: 10.

In some aspects, an anti-CD99 scFv can comprise a linker domain that connects the VL domain and the VH domain, wherein the linker domain comprises an amino acid sequence that is at least 97%, 98%, or 99% identical to SEQ ID NO: 11.

In some aspects, an anti-CD99 scFv can comprise a VH domain comprising the amino acid sequence of SEQ ID NO: 7. In some aspects, an anti-CD99 scFV can comprise a VL domain comprising the amino acid sequence of SEQ ID NO: 9. In some aspects, an anti-CD99 scFv can comprise a linker domain that connects the VL domain and the VH domain, wherein the linker domain comprises the amino acid sequence of SEQ ID NO: 11.

In some aspects, an anti-CD99 scFv can comprise, consist essentially of, or consist of an amino acid sequence that is at least 65%, 70%, 75%, 80%, 85%, 90%, 95%, 96%, 97%, 98%, 99% or 100% (or any percentage in between) identical to SEQ ID NO: 12. Accordingly, a nucleic acid sequence encoding for an anti-CD99 scFv can comprise, consist essentially of, or consist of a nucleic acid sequence that is at least 65%, 70%, 75%, 80%, 85%, 90%, 95%, 96%, 97%, 98%, 99% or 100% (or any percentage in between) identical to SEQ ID NO: 13.

### Chimeric Antigen Receptors of the Present Disclosure

The present disclosure provides chimeric antigen receptor (CAR) polypeptides comprising the anti-CD99 scFvs of the present disclosure.

A “chimeric antigen receptor” or is also known as an artificial cell receptor, a chimeric cell receptor, or a chimeric immunoreceptor. As would be appreciated by the skilled artisan, Chimeric antigen receptors (CARs) are engineered receptors, which graft a selected specificity onto an immune effector cell. CARs typically have an extracellular domain (ectodomain), a transmembrane domain and an intracellular (endodomain) domain. In some embodiments, the ectodomain comprises an antigen-binding domain and a hinge domain, wherein the antigen-binding domain specifically binds to an antigen that is of particular interest in the treatment of a specific disease or disorder (e.g. an antigen that is located on particular cancer cells or an antigen located on an infected cell). In some embodiments, the antigen is a protein expressed on the surface of cells (e.g., on the surface of a cancer cell, or an infected cell).

As would be appreciated by the skilled artisan, CARs are available in various different “formats”, sometimes also referred to as different “generations” of CARs (see e.g. Hiltensperger M, Krackhardt A M. Current and future concepts for the generation and application of genetically engineered CAR-T and TCR-T cells. Front Immunol. 2023 Mar. 6; 14:1121030. doi: 10.3389/fimmu.2023.1121030. PMID: 36949949; PMCID: PMC10025359; see also). Accordingly, the term CAR as used herein encompasses any of the formats/generations known in the art.

Briefly, first generation CARs provide a TCR-like signal from an Immunoreceptor Tyrosine-based Activation Motif (ITAM) containing intracellular signaling domain, most commonly derived from the CD3 zeta (CD3z or CD3ζ) molecule, and thereby elicit tumoricidal functions. However, the engagement of CD3 ζ-chain fusion receptors may not suffice to elicit substantial IL-2 secretion and/or T cell proliferation in the absence of a concomitant co-stimulatory signal. In physiological T cell responses, optimal lymphocyte activation requires the engagement of one or more co-stimulatory receptors such as CD28 or 4-1BB. In the setting of suboptimal activation elicited by first generation CARs, T cell activity in vivo is often transient and incapable of controlling the malignancy.

Second generation CARs have been constructed to transduce a functional antigen-dependent co-stimulatory signal in human primary T cells in addition to antigen-dependent TCR-like signal, permitting T cell proliferation in addition to tumoricidal activity. Second generation CARs most commonly provide co-stimulation using co-stimulatory domains (synonymously, co-stimulatory signaling regions) derived from CD28 or 4-1BB. The combined delivery of co-stimulation plus a CD3ζ signal renders 2^{nd} generation CARs superior in terms of function as compared to their first generation counterparts (CD3ζ signal alone). An example of a 2nd generation CAR is found in U.S. Pat. No. 7,446,190, incorporated herein by reference.

Third generation CARs have also been prepared. These combine multiple co-stimulatory domains (synonymously, co-stimulatory signaling regions) with a TCR-like signaling domain in cis, such as CD28+4-1BB+CD3ζ or CD28+OX40+CD3ζ, to further augment potency. In the 3^{rd} generation CARs, the co-stimulatory domains are aligned in series in the CAR intracellular domain and are generally placed upstream of CD3ζ or its equivalent. In general, however, the results achieved with these third generation CARs have been disappointing, showing only a marginal improvement over 2nd generation configurations, with some 3^{rd} generation CARs being inferior to 2^{nd} generation configurations.

Accordingly, the present disclosure provides various CARs that engage with CD99 via an antigen-binding domain that comprises the anti-CD99 scFvs of the present disclosure.

The present disclosure provides anti-CD99 CAR polypeptides comprising, consisting essentially of, or consisting of, from N-terminus to C-Terminus, an anti-CD99 antigen binding domain, a transmembrane, and an intracellular domain.

In some aspects, the anti-CD99 CAR polypeptides of the present disclosure can further comprise a hinge domain located between the anti-CD99 antigen binding domain and the transmembrane domain. Accordingly, the present disclosure provides anti-CD99 CAR polypeptides comprising, consisting essentially of, or consisting of, from N-terminus to C-terminus, an anti-CD99 antigen binding domain, a hinge domain, a transmembrane domain and an intracellular domain.

In some aspects, the anti-CD99 CAR polypeptides of the present disclosure can further comprise a signal peptide at the N-terminus of the anti-CD99 CAR polypeptide. Accordingly, the present disclosure provides anti-CD99 CAR polypeptides comprising, consisting essentially of, or consisting of, from N-terminus to C-terminus, a signal peptide, an anti-CD99 antigen binding domain, a transmembrane domain, and an intracellular domain. As described supra, the anti-CD99 CAR polypeptides of the present disclosure can also comprise a hinge domain. Accordingly, the present disclosure provides anti-CD99 CAR polypeptides comprising, consisting essentially of, or consisting of, from N-terminus to C-terminus, a signal peptide, an anti-CD99 antigen binding domain, a hinge domain, a transmembrane domain, and an intracellular domain.

As described supra, the anti-CD99 CAR polypeptides of the present disclosure can take the form of any of the CAR “generations” or formats known in the art. Accordingly, the intracellular domain of the anti-CD-99 CAR polypeptides can take any of the formats known in the art for intracellular domains of CAR polypeptides.

Intracellular domains of the anti-CD99 CAR polypeptides of the present disclosure can comprise at least one costimulatory domain, wherein the at least one costimulatory domain comprises the intracellular domain, or a fragment thereof, of a costimulatory molecule.

A “costimulatory molecule” as used herein refers to the cognate binding partner on an immune cell, e.g., a T cell, that specifically binds with a co-stimulatory ligand, thereby mediating a costimulatory response by the cell, such as, but not limited to proliferation. Costimulatory molecules include, but are not limited to an MHC class I molecule, BTLA and Toll ligand receptor. Examples of costimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like.

A “costimulatory ligand” refers to a molecule on an antigen presenting cell that specifically binds a cognate costimulatory signal molecule on an immune cell, e.g., a T cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1BBL, OX40L, inducible costimulatory igand (ICOS-L), intercellular adhesion molecule (ICAM, CD30L, CD40, CD70, CD83, HLA-G, MICA, MICB, HVEM, lymphotoxin β receptor, 3/TR6, ILT3, ILT4, an agonist or antibody that binds Toll ligand receptor and a ligand that specifically binds with B7-H3. A costimulatory ligand also encompasses, inter alia, an antibody that specifically binds with a co-stimulatory molecule present on a T cell, such as but not limited to, CD27, CD28, 4-1BB, OX40, CD30, CD40, PD-1, ICOS, lymphocyte function-associated antigen-1 (LFA-1), CD2, CD7, LTGHT, NKG2C, B7-H3, a ligand that specifically binds with CD83.

Intracellular domains of the anti-CD99 CAR polypeptides of the present disclose can comprise at least one activation domain comprising one or more cytoplasmic signaling sequences that initiate antigen-dependent primary activation. As would be apricated by the skilled artisan, primary cytoplasmic signaling sequences can comprise, consist essentially of, or consist of signaling motifs which are known as immunoreceptor tyrosine-based activation motifs (ITAMs). ITAMs are well defined signaling motifs found in the intracytoplasmic tail of a variety of receptors that serve as binding sites for syk/zap70 class tyrosine kinases. Examples of ITAMs, including, but are not limited to, those derived from TCRζ, FcRγ, FcRβ, FcRε, CD3γ, CD3δ, CD3ε, CD3ζ, CD5, CD22, CD79a, CD79b and CD66d.

In a non-limiting, an intracellular domain of an anti-CD99 CAR polypeptide of the present disclosure can comprise, consist essentially of, or consist of, from N-terminus to C-terminus, a costimulatory domain, and an activation domain. Accordingly, the present disclosure provides anti-CD99 polypeptides comprising, consisting essentially of, or consisting of, from N-terminus to C-terminus, a signal peptide, an anti-CD99 antigen binding domain, a hinge domain, a transmembrane domain, and an activation domain. In some aspects, the activation domain comprises a CD3ζ activation polypeptide. In some aspects, the costimulatory domain comprises a 4-1BB costimulatory polypeptide. In some aspects, the costimulatory domain comprises a CD28 costimulatory polypeptide.

In another non-limiting example, an intracellular domain of an anti-CD99 CAR polypeptide of the present disclosure can comprise, consist essentially of, or consist of, from N-terminus to C-terminus, a first costimulatory domain, a second costimulatory domain, and an activation domain. In some aspects, the activation domain comprises a CD3ζ activation polypeptide. In some aspects, the first costimulatory domain comprises at least one of a 4-1BB costimulatory polypeptide and a CD28 costimulatory polypeptide. In some aspects, the second costimulatory domain comprises at least one of a 4-1BB costimulatory polypeptide, a CD27 costimulatory polypeptide, an ICOS costimulatory domain, and an OX40 costimulatory domain.

The following are exemplary anti-CD99 CAR polypeptides of the present disclosure.

The present disclosure provides anti-CD99 CAR polypeptides comprising, consisting essentially of, or consisting of, from N-terminus to C-terminus, a signal peptide, an anti-CD99 antigen binding domain comprising an anti-CD99 scFv, a hinge domain comprising a CD8 hinge polypeptide, a transmembrane domain comprising a CD8 transmembrane polypeptide, a costimulatory domain comprising a 4-1BB costimulatory polypeptide, and an activation domain comprising a CD3 activation polypeptide. In some aspects of the preceding anti-CD99 CAR polypeptides, the signal peptide can comprise a CD8 signal peptide.

The present disclosure provides anti-CD99 CAR polypeptides comprising, consisting essentially of, or consisting of, from N-terminus to C-terminus, a signal peptide, an anti-CD99 antigen binding domain comprising an anti-CD99 scFv, wherein the anti-CD99 scFv comprises an amino acid sequence that is at least 97%, 98%, or 99% identical to SEQ ID NO: 12, a hinge domain comprising a CD8 hinge polypeptide, wherein the CD8 hinge polypeptide comprises an amino acid sequence that is at least 97%, 98%, or 99% identical to SEQ ID NO: 16, a transmembrane domain comprising a CD8 transmembrane polypeptide, wherein the CD8 transmembrane polypeptide comprises an amino acid sequence that is at least 97%, 98%, 99% identical to SEQ ID NO: 20, a costimulatory domain comprising a 4-1BB costimulatory polypeptide, wherein the 4-1BB costimulatory polypeptide comprises an amino acid sequence that is at least 97%, 98%, or 99% identical to SEQ ID NO: 24, and an activation domain comprising a CD3ζ activation polypeptide, wherein the CD3ζ activation polypeptide comprises an amino acid sequence that is at least 97%, 98%, or 99% identical to SEQ ID NO: 26. In some aspects of the preceding anti-CD99 CAR polypeptides, the signal peptide can comprise a CD8 signal peptide, wherein the CD8 signal peptide comprises an amino acid sequence that is at least 97%, 98%, or 99% identical to SEQ ID NO: 28.

The present disclosure provides anti-CD99 CAR polypeptides comprising, consisting essentially of, or consisting of, from N-terminus to C-terminus, a signal peptide, an anti-CD99 antigen binding domain comprising an anti-CD99 scFv, wherein the anti-CD99 scFv comprises the amino acid sequence of SEQ ID NO: 12, a hinge domain comprising a CD8 hinge polypeptide, wherein the CD8 hinge polypeptide comprises the amino acid sequence of SEQ ID NO: 16, a transmembrane domain comprising a CD8 transmembrane polypeptide, wherein the CD8 transmembrane polypeptide comprises the amino acid sequence of SEQ ID NO: 20, a costimulatory domain comprising a 4-1BB costimulatory polypeptide, wherein the 4-1BB costimulatory polypeptide comprises the amino acid sequence of SEQ ID NO: 24, and an activation domain comprising a CD3ζ activation polypeptide, wherein the CD3ζ activation polypeptide comprises the amino acid sequence of SEQ ID NO: 26. In some aspects of the preceding anti-CD99 CAR polypeptides, the signal peptide can comprise a CD8 signal peptide, wherein the CD8 signal peptide comprises the amino acid sequence of SEQ ID NO: 28.

In some aspects, a an anti-CD99 CAR polypeptide can comprise, consist essentially of, or consist of an amino acid sequence that is at least 65%, 70%, 75%, 80%, 85%, 90%, 95%, 96%, 97%, 98%, 99% or 100% (or any percentage in between) identical to SEQ ID NO: 32. Accordingly, a nucleic acid sequence encoding for an anti-CD99 CAR polypeptide can comprise, consist essentially of, or consist of a nucleic acid sequence that is at least 65%, 70%, 75%, 80%, 85%, 90%, 95%, 96%, 97%, 98%, 99% or 100% (or any percentage in between) identical to SEQ ID NO: 33.

The present disclosure provides anti-CD99 CAR polypeptides comprising, consisting essentially of, or consisting of, from N-terminus to C-terminus, a signal peptide, an anti-CD99 antigen binding domain comprising an anti-CD99 scFv, a hinge domain comprising a CD28 hinge polypeptide, a transmembrane domain comprising a CD28 transmembrane polypeptide, a costimulatory domain comprising a CD28 costimulatory polypeptide, and an activation domain comprising a CD3ζ activation polypeptide. In some aspects of the preceding anti-CD99 CAR polypeptides, the signal peptide can comprise a CD8 signal peptide.

The present disclosure provides anti-CD99 CAR polypeptides comprising, consisting essentially of, or consisting of, from N-terminus to C-terminus, a signal peptide, an anti-CD99 antigen binding domain comprising an anti-CD99 scFv, wherein the anti-CD99 scFv comprises an amino acid sequence that is at least 97%, 98%, or 99% identical to SEQ ID NO: 12, a hinge domain comprising a CD28 hinge polypeptide, wherein the CD28 hinge polypeptide comprises an amino acid sequence that is at least 97%, 98%, or 99% identical to SEQ ID NO: 14, a transmembrane domain comprising a CD28 transmembrane polypeptide, wherein the CD28 transmembrane polypeptide comprises an amino acid sequence that is at least 97%, 98%, 99% identical to SEQ ID NO: 18, a costimulatory domain comprising a CD28 costimulatory polypeptide, wherein the CD28 costimulatory polypeptide comprises an amino acid sequence that is at least 97%, 98%, or 99% identical to SEQ ID NO: 22, and an activation domain comprising a CD3ζ activation polypeptide, wherein the CD3ζ activation polypeptide comprises an amino acid sequence that is at least 97%, 98%, or 99% identical to SEQ ID NO: 26. In some aspects of the preceding anti-CD99 CAR polypeptides, the signal peptide can comprise a CD8 signal peptide, wherein the CD8 signal peptide comprises an amino acid sequence that is at least 97%, 98%, or 99% identical to SEQ ID NO: 28.

The present disclosure provides anti-CD99 CAR polypeptides comprising, consisting essentially of, or consisting of, from N-terminus to C-terminus, a signal peptide, an anti-CD99 antigen binding domain comprising an anti-CD99 scFv, wherein the anti-CD99 scFv comprises the amino acid sequence of SEQ ID NO: 12, a hinge domain comprising a CD28 hinge polypeptide, wherein the CD28 hinge polypeptide comprises the amino acid sequence of SEQ ID NO: 14, a transmembrane domain comprising a CD28 transmembrane polypeptide, wherein the CD28 transmembrane polypeptide comprises the amino acid sequence of SEQ ID NO: 18, a costimulatory domain comprising a CD28 costimulatory polypeptide, wherein the CD28 costimulatory polypeptide comprises the amino acid sequence of SEQ ID NO: 22, and an activation domain comprising a CD3ζ activation polypeptide, wherein the CD3ζ activation polypeptide comprises the amino acid sequence of SEQ ID NO: 26. In some aspects of the preceding anti-CD99 CAR polypeptides, the signal peptide can comprise a CD8 signal peptide, wherein the CD8 signal peptide comprises the amino acid sequence of SEQ ID NO: 28.

In some aspects, a an anti-CD99 CAR polypeptide can comprise, consist essentially of, or consist of an amino acid sequence that is at least 65%, 70%, 75%, 80%, 85%, 90%, 95%, 96%, 97%, 98%, 99% or 100% (or any percentage in between) identical to SEQ ID NO: 34. Accordingly, a nucleic acid sequence encoding for an anti-CD99 CAR polypeptide can comprise, consist essentially of, or consist of a nucleic acid sequence that is at least 65%, 70%, 75%, 80%, 85%, 90%, 95%, 96%, 97%, 98%, 99% or 100% (or any percentage in between) identical to SEQ ID NO: 35.

### Transmembrane Domains

The anti-CD99 CAR polypeptides of the present disclosure are intended to be expressed on the surface membrane of the cell. Accordingly, the anti-CD99 CAR polypeptides of the present disclosure can comprise a transmembrane domain.

As would be appreciated by the skilled artisan, a transmembrane domain may be any protein structure which is thermodynamically stable in a membrane. This is typically an alpha helix comprising of several hydrophobic residues. The transmembrane domain of any transmembrane protein can be used to supply the transmembrane portion of the anti-CD99 CAR polypeptides of the present disclosure. The presence and span of a transmembrane domain of a protein can be determined by those skilled in the art using the DeepTMHMM algorithm (see Hallgren et al. bioRxiv (2022). DeepTMHMM predicts alpha and beta transmembrane proteins using deep neural networks. https://doi.org/10.1101/2022.04.08.487609), or any equivalent algorithm known in the art.

Transmembrane domains can be derived either from a natural or from a synthetic source. Transmembrane domains can be derived from any membrane-bound or transmembrane protein. As non-limiting examples, the transmembrane polypeptide can be a subsequence or subunit of the T cell receptor such as α, β, γ or δ, polypeptide constituting CD3 complex, IL-2 receptor p55 (α chain), p75 (β chain) or γ chain, subunit chain of Fc receptors, in particular Fcγ receptor III or CD proteins. Alternatively, transmembrane domains can be synthetic and can comprise, consist essentially of, or consist of hydrophobic residues such as leucine and valine.

In some aspects, a transmembrane domain can comprise, consist essentially of, or consist of a CD8 transmembrane domain.

In some aspects, a CD8 transmembrane domain can comprise, consist essentially of, or consist of an amino acid sequence that is at least 65%, 70%, 75%, 80%, 85%, 90%, 95%, 96%, 97%, 98%, 99% or 100% (or any percentage in between) identical to SEQ ID NO: 20. Accordingly, a nucleic acid sequence encoding for a CD8 transmembrane domain can comprise, consist essentially of, or consist of a nucleic acid sequence that is at least 65%, 70%, 75%, 80%, 85%, 90%, 95%, 96%, 97%, 98%, 99% or 100% (or any percentage in between) identical to SEQ ID NO: 21.

In some aspects, a transmembrane domain can comprise, consist essentially of, or consist of a CD28 transmembrane domain.

In some aspects, a CD28 transmembrane domain can comprise, consist essentially of, or consist of an amino acid sequence that is at least 65%, 70%, 75%, 80%, 85%, 90%, 95%, 96%, 97%, 98%, 99% or 100% (or any percentage in between) identical to SEQ ID NO: 18. Accordingly, a nucleic acid sequence encoding for CD28 transmembrane domain can comprise, consist essentially of, or consist of a nucleic acid sequence that is at least 65%, 70%, 75%, 80%, 85%, 90%, 95%, 96%, 97%, 98%, 99% or 100% (or any percentage in between) identical to SEQ ID NO: 19.

In some aspects, a costimulatory domain can comprise, consist essentially of, or consist of the intracellular domain, or a fragment thereof, of 4-1BB (CD137). Such costimulatory domains are referred to herein as 4-1BB costimulatory polypeptides. Accordingly, a costimulatory domain of a CAR polypeptide of the present disclosure can comprise, consist essentially of, or consist of at least one 4-1BB costimulatory polypeptide.

In some aspects, a 4-1BB costimulatory polypeptide can comprise, consist essentially of, or consist of an amino acid sequence that is at least 65%, 70%, 75%, 80%, 85%, 90%, 95%, 96%, 97%, 98%, 99% or 100% (or any percentage in between) identical to SEQ ID NO: 24. Accordingly, a nucleic acid sequence encoding for a 4-1BB costimulatory polypeptide can comprise, consist essentially of, or consist of a nucleic acid sequence that is at least 65%, 70%, 75%, 80%, 85%, 90%, 95%, 96%, 97%, 98%, 99% or 100% (or any percentage in between) identical to SEQ ID NO: 25.

In some aspects, a costimulatory domain can comprise, consist essentially of, or consist of the intracellular domain, or a fragment thereof of, CD28. Such costimulatory domains are referred to herein as CD28 costimulatory polypeptides. Accordingly, a costimulatory domain of a CAR polypeptide of the present disclosure can comprise, consist essentially of, or consist of at least one CD28 costimulatory polypeptide.

In some aspects, a CD28 costimulatory polypeptide can comprise, consist essentially of, or consist of an amino acid sequence that is at least 65%, 70%, 75%, 80%, 85%, 90%, 95%, 96%, 97%, 98%, 99% or 100% (or any percentage in between) identical to SEQ ID NO: 22. Accordingly, a nucleic acid sequence encoding for a CD28 costimulatory polypeptide can comprise, consist essentially of, or consist of a nucleic acid sequence that is at least 65%, 70%, 75%, 80%, 85%, 90%, 95%, 96%, 97%, 98%, 99% or 100% (or any percentage in between) identical to SEQ ID NO: 23.

In some aspects, an activation domain can comprise, consist essentially of, or consist of a CD3ζ activation polypeptide.

In some aspects, a CD3ζ activation polypeptide can comprise, consist essentially of, or consist of an amino acid sequence that is at least 65%, 70%, 75%, 80%, 85%, 90%, 95%, 96%, 97%, 98%, 99% or 100% (or any percentage in between) identical to SEQ ID NO: 26. Accordingly, a nucleic acid sequence encoding for a CD3ζ activation polypeptide can comprise, consist essentially of, or consist of a nucleic acid sequence that is at least 65%, 70%, 75%, 80%, 85%, 90%, 95%, 96%, 97%, 98%, 99% or 100% (or any percentage in between) identical to SEQ ID NO: 27.

### Hinge Domains

In some aspects, a hinge domain of a CAR polypeptide of the present disclosure refers to a polypeptide that is located between the antigen binding domain and the transmembrane domain. Without wishing to be bound by theory, a hinge domain can provide more flexibility and accessibility for the antigen binding domain.

In some aspects, a hinge domain may comprise, consist essentially or, or consist of up to 300 amino acids.

In some aspects, a hinge domain can comprise, consist essentially or, or consist of 10 to 100 amino acids or 25 to 50 amino acids.

In some aspects, a hinge domain can be derived from all or part of naturally occurring molecules, such as from all or part of the extracellular region of CD8, CD4, CD28, 4-1BB, or IgG (in particular, the hinge region of an IgG), or from all or part of an antibody heavy-chain constant region.

In some aspects, a hinge domain can be a synthetic sequence that corresponds to a naturally occurring hinge sequence, or may be an entirely synthetic hinge sequence. In some aspects, a hinge domain can comprise, consist essentially or, or consist of a subsequence of CD8a, an IgG1, or an FcγRIIIα.

In some aspects, a hinge domain can comprise, consist essentially of, or consist of a CD28 hinge polypeptide.

In some aspects, a CD28 hinge polypeptide can comprise, consist essentially of, or consist of an amino acid sequence that is at least 65%, 70%, 75%, 80%, 85%, 90%, 95%, 96%, 97%, 98%, 99% or 100% (or any percentage in between) identical to SEQ ID NO: 14. Accordingly, a nucleic acid sequence encoding for a CD28 hinge polypeptide can comprise, consist essentially of, or consist of a nucleic acid sequence that is at least 65%, 70%, 75%, 80%, 85%, 90%, 95%, 96%, 97%, 98%, 99% or 100% (or any percentage in between) identical to SEQ ID NO: 15.

In some aspect, a hinge domain can comprise, consist essentially of, or consist of a CD8 hinge polypeptide.

In some aspects, a CD8 hinge polypeptide can comprise, consist essentially of, or consist of an amino acid sequence that is at least 65%, 70%, 75%, 80%, 85%, 90%, 95%, 96%, 97%, 98%, 99% or 100% (or any percentage in between) identical to SEQ ID NO: 16. Accordingly, a nucleic acid sequence encoding for a CD8 hinge polypeptide can comprise, consist essentially of, or consist of a nucleic acid sequence that is at least 65%, 70%, 75%, 80%, 85%, 90%, 95%, 96%, 97%, 98%, 99% or 100% (or any percentage in between) identical to SEQ ID NO: 17.

### Signal Peptides

As used herein, the term “signal peptide” refers to a peptide which functions to direct the polypeptide to which its attached to the secretory pathway. As would be appreciated by the skilled artisan, in contexts wherein a polypeptide of the present disclosure comprises both a signal peptide and a transmembrane domain, the signal peptide functions to direct that polypeptide to the secretory pathway such that the polypeptide eventually embedded within a membrane, preferably the cellular membrane. Signal peptides are well known in the art. Accordingly, a signal peptide within the polypeptides of the present disclosure can be any signal peptide known in the art that is sufficient to direct the polypeptide to which its attached to the secretory pathway.

In some aspects, a signal peptide can comprise, consist essentially of, or consist of a CD8 signal peptide.

In some aspects, a CD8 signal peptide can comprise, consist essentially of, or consist of an amino acid sequence that is at least 65%, 70%, 75%, 80%, 85%, 90%, 95%, 96%, 97%, 98%, 99% or 100% (or any percentage in between) identical to SEQ ID NO: 28. Accordingly, a nucleic acid sequence encoding for a CD8 signal peptide can comprise, consist essentially of, or consist of a nucleic acid sequence that is at least 65%, 70%, 75%, 80%, 85%, 90%, 95%, 96%, 97%, 98%, 99% or 100% (or any percentage in between) identical to SEQ ID NO: 29.

In some aspects, a signal peptide can comprise, consist essentially of, or consist of a GMCSF signal peptide.

In some aspects, a GMCSF signal peptide can comprise, consist essentially of, or consist of an amino acid sequence that is at least 65%, 70%, 75%, 80%, 85%, 90%, 95%, 96%, 97%, 98%, 99% or 100% (or any percentage in between) identical to SEQ ID NO: 30. Accordingly, a nucleic acid sequence encoding for a GMCSF signal peptide can comprise, consist essentially of, or consist of a nucleic acid sequence that is at least 65%, 70%, 75%, 80%, 85%, 90%, 95%, 96%, 97%, 98%, 99% or 100% (or any percentage in between) identical to SEQ ID NO: 31.

### Functional Variants of the Polypeptides of the Present Disclosure

The present disclosure also contemplates functional variants of the anti-CD99 scFvs or CAR polypeptides disclosed and described herein. The term “functional variant” as used herein refers to an anti-CD99 scFv or CAR polypeptide having substantial or significant sequence identity or similarity to a parent anti-CD99 scFv or CAR where the functional variant retains the biological activity of the parent anti-CD99 scFv or CAR polypeptide of which it is a variant. Functional variants encompass, for example, those variants of the anti-CD99 scFv or CAR polypeptide described herein (the parent anti-CD99 scFv or CAR polytpeptide) that retain the ability to recognize target cells to a similar extent, the same extent, or to a higher extent, as the parent anti-CD99 scFv or CAR polypeptide. In reference to the anti-CD99 scFv or parent CAR the functional variant can, for instance, be at least about 90%, about 91%, about 92%, about 93%, about 94%, about 95%, about 96%, about 97%, about 98%, about 99% or more identical in amino acid sequence to the parent CAR.

A functional variant can, for example, comprise the amino acid sequence of the anti-CD99 scFv or parent CAR with at least one conservative amino acid substitution. Alternatively, or additionally, the functional variants can comprise the amino acid sequence of the parent anti-CD99 scFv or CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent anti-CD99 scFv or CAR.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of +2 are substituted. The hydrophilicity of amino acids can also be used to reveal substitutions that would result in proteins retaining biological function. A consideration of the hydrophilicity of amino acids in the context of a polypeptide permits calculation of the greatest local average hydrophilicity of that polypeptide, a useful measure that has been reported to correlate well with antigenicity and immunogenicity. U.S. Pat. No. 4,554,101, incorporated fully herein by reference.

Substitution of amino acids having similar hydrophilicity values can result in polypeptides retaining biological activity, for example immunogenicity. Substitutions can be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Tables A, B, or C below. In some aspects, fusion polypeptides and/or nucleic acids encoding such fusion polypeptides include conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Exemplary conservative substitutions are set out in Table A.

TABLE A
Conservative Substitutions I

| Side chain characteristics | | Amino Acid |
|---|---|---|
| Aliphatic | Non-polar | G A P I L V F |
| | Polar - uncharged | C S T M N Q |
| | Polar - charged | D E K R |

| | |
|---|---|
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| Side Chain Characteristic | | Amino Acid |
|---|---|---|
| Non-polar | Aliphatic: | A L I V P |
| (hydrophobic) | Aromatic: | F W Y |
| | Sulfur-containing: | M |
| | Borderline: | G Y |
| Uncharged-polar | Hydroxyl: | S T Y |
| | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, exemplary conservative substitutions are set out in Table C.

TABLE C
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides of the disclosure are intended to include polypeptides bearing one or more insertions, deletions, or substitutions, or any combination thereof, of amino acid residues as well as modifications other than insertions, deletions, or substitutions of amino acid residues. Polypeptides or nucleic acids of the disclosure may contain one or more conservative substitution.

As used throughout the disclosure, the term “more than one” of the aforementioned amino acid substitutions refers to 2, 3, 4, 5, 6, 7, 8, 9, 10, 11, 12, 13, 14, 15, 16, 17, 18, 19, or 20 or more of the recited amino acid substitutions. The term “more than one” may refer to 2, 3, 4, or 5 of the recited amino acid substitutions.

### Nucleic Acid Molecules of the Present Disclosure

The present disclosure provides nucleic acid molecules comprising, consisting essentially of, or consisting of one or more nucleic acid sequences encoding for an anti-CD99 CAR polypeptide of the present disclosure.

The present disclosure provides nucleic acid molecules comprising a nucleic acid sequence that comprises, consists essentially or, or consist of a nucleic acid sequence that is at least 65%, 70%, 75%, 80%, 85%, 90%, 95%, 96%, 97%, 98%, 99% or 100% (or any percentage in between) identical to SEQ ID NO: 33.

The present disclosure provides nucleic acid molecules comprising a nucleic acid sequence that comprises, consists essentially or, or consist of a nucleic acid sequence that is at least 65%, 70%, 75%, 80%, 85%, 90%, 95%, 96%, 97%, 98%, 99% or 100% (or any percentage in between) identical to SEQ ID NO: 35.

The present disclosure also provides vectors comprising at least one nucleic acid molecule of the present disclosure. In some aspects, the vector can be a viral vector. In some aspects, the viral vector is an AAV vector or a lentiviral vector.

The present disclosure also encompasses all nucleic acid molecules that are complementary to the nucleic acid molecules described in detail herein.

### Cells

The present disclosure provides cells that express at least one anti-CD99 CAR polypeptide of the present disclosure. These cells that express at least one anti-CD99 CAR polypeptide of the present disclosure are also referred to herein as “therapeutic cells” as they can be used in methods of treating cancer and preventing cancer metastasis, as described infra.

Accordingly, the present disclosure provides populations (also referred to herein as pluralities) of cells expressing at least one anti-CD99 CAR polypeptide of the present disclosure.

The present disclosure provides populations of cells, wherein at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 97%, or at least about 99% of the cells in the plurality express an anti-CD99 CAR polypeptide of the present disclosure.

In some aspects, the cells described above can be immune cells.

As used herein, “immune cell” refers to a cell of hematopoietic origin functionally involved in the initiation and/or execution of innate and/or adaptative immune response.

Examples of immune cells include, but are not limited to, T cells (e.g., regulatory T cells, CAR T cells, CD8+ CAR T cells, CD4+ CAR T cells, CD4+ T cells, CD8+ T cells, peripheral blood (PB) derived T cells, umbilical cord blood (UCB) derived T cells, or gamma-delta T cells), NK cells, NK-like cells, invariant NK cells, NKT cells, cytokine induced killer (CIK) cells, stem cells (e.g., mesenchymal stem cells (MSCs), hematopoietic stem cells, hematopoietic progenitor cells, or induced pluripotent stem (iPSC) cells). In some embodiments, the cells are monocytes or granulocytes, e.g., myeloid cells, macrophages, neutrophils, dendritic cells, mast cells, eosinophils, and/or basophils.

In some aspects, the cells are T cells.

In some aspects, the cells are NK cells.

In some aspects, the cells described above can be tumor infiltrating lymphocytes (“TILs”). As would be appreciated by the skilled artisan, TILs refers to populations of white blood cells that have left the bloodstream of a subject and migrated into a tumor. Populations of TILs can include, but are not limited to, T helper 17 cells (Thl7, CD4+IL17+ T cells), cytotoxic T cells (Tc17, CD8+IL17+ T cells) and regulatory T cells (Treg, CD4+CD25+Foxp3+ T cells), natural killer (NK) cells, dendritic cells and MI macrophages. As would be appreciated by the skilled artisan, TILs can generally be defined either biochemically, using cell surface markers, or functionally, by their ability to infiltrate tumors and effect treatment. TILs can be generally categorized by expressing one or more of the following biomarkers: CD4, CD8, TCR αβ, CD27, CD28, CD56, CCR7, CD45Ra, CD95, PD-1, and CD25. Additionally and alternatively, TILs can be functionally defined by their ability to infiltrate solid tumors upon reintroduction into a subject.

Immune cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the immune cells are enriched, isolated, and/or purified may be isolated from both living and non-living subjects, wherein the non-living subjects are organ donors. The isolated immune cells may be used directly, or they can be stored for a period of time, such as by freezing. In the case of TILs, the population of TILs can be enriched/purified from a tumor sample obtained from the subject.

In some aspects, the cells of the present disclosure that express an anti-CD99 CAR polypeptides can be modified such that the cells do not express endogenous CD99. That is, the cells of the present disclosure can be genetically modified to “knock-out” endogenous CD99.

Accordingly, the present disclosure provides cells expressing an anti-CD99 CAR polypeptides of the present disclosure, wherein the cells do not express CD99. In some aspects, these cells are T-cells. In some aspects, these cells are NK cells.

Accordingly, the present disclosure provides populations of cells expressing at least one anti-CD99 CAR polypeptide of the present disclosure, wherein the cells in the population do not express CD99.

The present disclosure provides populations of cells, wherein at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 97%, or at least about 99% of the cells in the plurality express an anti-CD99 CAR polypeptide of the present disclosure and do not express CD99.

The present disclosure also provides pharmaceutical compositions comprising the therapeutic cells or pluralities of therapeutic cells described herein. In some aspects, the pharmaceutical compositions further comprise one or more pharmaceutically acceptable carriers, diluents or excipients. The pharmaceutical compositions of the present disclosure can further comprise one or more further pharmaceutically active polypeptides and/or compounds. The pharmaceutical compositions of the present disclosure can be formulated for a particular administration route, for example, intravenous infusion or intrathecal infusion.

As used herein, “pharmaceutically acceptable carrier,” “pharmaceutical acceptable excipient,” and “pharmaceutically acceptable diluents” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Exemplary diluents for aerosol or parenteral administration are phosphate buffered saline (PBS) or normal (0.9%) saline. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 21st Ed. Mack Publishing, 2005).

### Methods of Use

The present disclosure provides methods of treating cancer in a subject, the method comprising administering to the subject one or more pluralities of therapeutic cells of the present disclosure.

The present disclosure provides one or more pluralities of therapeutic cells of the present disclosure for use in the treatment of cancer in a subject.

The present disclosure provides the use of one or more pluralities of therapeutic cells of the present disclosure for use in the manufacture of a medicament for the treatment of cancer.

The present disclosure provides methods of preventing cancer metastasis in a subject, the method comprising administering to the subject one or more pluralities of therapeutic cells of the present disclosure.

The present disclosure provides one or more pluralities of therapeutic cells of the present disclosure for use in the prevention of cancer metastasis in a subject.

The present disclosure provides the use of one or more pluralities of therapeutic cells of the present disclosure for use in the manufacture of a medicament for the prevention of cancer metastasis.

In some aspects, the plurality or pluralities of therapeutic cells are administered to the subject in a therapeutically effective amount.

In some aspects, the plurality or pluralities of therapeutic cells are administered by intravenous administration. In some aspects, the plurality or pluralities of therapeutic cells are administered by intrathecal administration. In some aspects, the plurality or pluralities of therapeutic cells are administered by intratumoral administration. In some aspects, the plurality or pluralities of therapeutic cells are administered by intrapleural administration, intraperitoneal administration, or intrathoracic administration.

The present disclosure provides methods of treating cancer in the subject, the method comprising administering to the subject one or more pluralities of therapeutic cells of the present disclosure and at least one additional therapy. In some aspects, the at least one additional therapy can be selected from immunotherapy, a stem cell transplant, an anti-cancer therapy, chemotherapy, targeted drug therapy, radiation therapy, or any combination thereof. In some aspects, the at least one additional therapy is radiation therapy. In some aspects, the at least one additional therapy is chemotherapy. In some aspects, the at least one additional therapy comprises the administration of a combination of venetoclax and azacitidine. In some aspects, the at least one additional therapy is surgery. In some aspects, the subject can be pre-treated with radiation therapy prior to receiving the therapeutic cells of the present disclosure.

In some aspects, the at least one additional therapy and the one or more pluralities of therapeutic cells of the present disclosure can be administered in temporal proximity.

As used herein, the term “temporal proximity” refers to that administration of one therapeutic agent (e.g., one or more pluralities of therapeutic cells of the present disclosure) occurs within a time period before or after the administration of another therapeutic agent (e.g., radiation therapy), such that the therapeutic effect of the one therapeutic agent overlaps with the therapeutic effect of the other therapeutic agent. In some embodiments, the therapeutic effect of the one therapeutic agent completely overlaps with the therapeutic effect of the other therapeutic agent. In some embodiments, “temporal proximity” means that administration of one therapeutic agent occurs within a time period before or after the administration of another therapeutic agent, such that there is a synergistic effect between the one therapeutic agent and the other therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

The terms “cancer” and “cancerous” refer to or describe the physiological condition in mammals that is typically characterized by unregulated cell growth.

In some aspects, the cancer can be a solid tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast.

In some aspects, the cancer is a hematological cancer. Exemplary hematological tumors include but are not limited to tumors of the bone marrow, T or B cell malignancies, myeloid malignancies, leukemias, lymphomas, blastomas, myelomas.

Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma;

lymphoepithelial carcinoma; basal cell carcinoma; pilomatrix carcinoma; transitional cell carcinoma; papillary transitional cell carcinoma; adenocarcinoma; gastrinoma, malignant;

cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; T lymphoblastic leukemia; T lymphoblastic lymphoma; B cell leukaemia; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; B cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); chronic myeloid leukemia, acute lymphoblastic leukemia (ALL); acute lymphoblastic lymphoma; acute myeloid leukemia (AML); myelodysplastic syndrome (MDS); myeloproliferative neoplasms; chronic myeloblasts leukemia; diffuse large B cell lymphoma (DLBCL); peripheral T cell lymphoma (PTCL); or anaplastic large cell lymphoma (ALCL). In some embodiments, the cancer comprises a liquid tumor. In some embodiments, the liquid tumor is a leukemia or a lymphoma. In some embodiments, the leukemia or lymphoma is B cell leukemia or B cell lymphoma.

In some aspects, the cancer is a cancer that is characterized by expression of CD99 in the cancerous cells.

In some aspects, the cancer is diffuse intrinsic pontine glioma (DIPG).

In some aspects, the cancer is acute myeloid leukemia (AML).

In some aspects, the cancer is Ewing Sarcoma.

In some aspects, the cancer is an NK/T-cell lymphoma.

In some aspects, the cancer is a large granular lymphocyte leukemia.

In some aspects, the cancer is a chronic myeloid leukemia

In some aspects, the cancer is a glioblastoma.

In some aspects, the cancer is a glioma.

In some aspects, the cancer is an ependymoma.

In some aspects, the cancer is an atypical teratoid/rhabdoid tumor.

In some aspects, the cancer is a neuroblastoma.

In some aspects, the cancer is glioblastoma multiforme (GBM). In some aspects, the subject is an adult who has GBM.

In some aspects, the cancer is recurrent cancer. Accordingly, the cancer can be selected from recurrent DIPG, recurrent AML, and recurrent Ewing Sarcoma.

In some aspects, the cancer is refractory to treatment with a treatment that is different from the therapeutic cells of the present disclosure. Accordingly, the cancer can be selected from refractory DIPG, refractory AML, and refractory Ewing Sarcoma.

In some aspects, the cancer has metastasized. Accordingly, the cancer can be selected from metastasized DIPG, metastasized AML, and metastasized Ewing Sarcoma.

The terms “subject” and “patient” are used interchangeably herein. In some embodiments, the subject treated in accordance with the methods described herein is a human patient. In some aspects, the subject is male. In some aspects, the subject is female.

In some aspects of the methods of the present disclosure, a subject can be at least about 5 years of age, or at least about 10 years of age, or at least about 15 years of age, or at least about 18 years of age, or at least about 20 years of age, or at least about 25 years of age, or at least about 30 years of age, or at least about 35 years of age, or at least about 40 years of age, or at least about 45 years of age, or at least about 50 years of age, or at least about 55 years of age, or at least about 60 years of age, or at least about 65 years of age, or at least about 70 years of age, or at least about 75 years of age, or at least about 80 years of age, or at least about 85 years of age, or at least about 90 years of age, or at least about 95 years of age, or at least about 100 years of age.

In some aspects of the methods of the present disclosure, a subject can be no more than about 5 years of age, or about 10 years of age, or about 15 years of age, or about 18 years of age, or about 20 years of age, or about 25 years of age, or about 30 years of age, or about 35 years of age, or about 40 years of age, or about 45 years of age, or about 50 years of age, or about 55 years of age, or about 60 years of age, or about 65 years of age, or about 70 years of age, or about 75 years of age, or about 80 years of age, or about 85 years of age, or about 90 years of age, or about 95 years of age, or about 100 years of age.

In some aspects of the methods of the present disclosure, a subject can be about 1 year of age to about 20 years of age, or about 2 years of age to about 10 years of age, or about 5 years of age to about 9 years of age, or about 10 years of age to about 15 years of age.

In some aspects, the subject can have received at least one previous therapy. That is, prior to being administered therapeutic cells of the present disclosure, the subject has received at least one previous therapy. In some aspects, the subject can be nonresponsive to the at least one previous therapy. In some aspects, the subject can have been initially responsive to the at least one previous therapy, but then became unresponsive over the course of treatment the at least one previous therapy. In some aspects, the at least one previous therapy is continued to be administered to the subject after the administration of therapeutic cells of the present disclosure.

In some aspects, the at least one previous therapy can be selected from surgery, an immunotherapy, a stem cell transplant, an anti-cancer therapy, chemotherapy, targeted drug therapy, radiation therapy, or any combination thereof. In some aspects, the at least one previous therapy is radiation therapy. In some aspects, the at least one previous therapy is chemotherapy. In some aspects, the at least one previous therapy comprises the administration of a combination of venetoclax and azacitidine.

In some aspects, the at least one previous therapy is surgery. In some aspects, the surgery comprises the amputation of one or more body parts. Accordingly, the present disclosure provides methods of preventing cancer metastasis in a subject having Ewing sarcoma, wherein the subject previously underwent an amputation of one or more body parts to treat the Ewing sarcoma.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

As used herein, “prevent”. “preventing” and the like describe stopping the onset of the disease, condition or disorder, or one or more symptoms or complications thereof.

The terms “effective amount” and “therapeutically effective amount” of cells, an agent, or a compound are used in the broadest sense to refer to a nontoxic but sufficient amount of the cells, active agent, or compound to provide the desired effect or benefit.

In some aspects, the therapeutic cells of the present disclosure can be administered to the subject in an amount of at least about 0.1×10⁶ cells/kg, or at least about 0.25×10⁶ cells/kg, or at least about 0.5×10⁶ cells/kg, or at least about 0.75×10⁶ cells/kg, or at least about 1×10⁶ cells/kg, or at least about 1.25×10⁶ cells/kg, or at least about 1.5×10⁶ cells/kg, or at least about 1.75×10⁶ cells/kg, or at least about 2×10⁶ cells/kg, or at least about 2.25×10⁶ cells/kg, or at least about 2.5×10⁶ cells/kg, or at least about 2.75×10⁶ cells/kg, or at least about 3×10⁶ cells/kg.

In some aspects, the therapeutic cells of the present disclosure can be administered to the subject in an amount of about 0.1×10⁶ cells/kg, or about 0.25×10⁶ cells/kg, or about 0.5×10⁶ cells/kg, or about 0.75×10⁶ cells/kg, or about 1×10⁶ cells/kg, or about 1.25×10⁶ cells/kg, or about 1.5×10⁶ cells/kg, or about 1.75×10⁶ cells/kg, or about 2×10⁶ cells/kg, or about 2.25×10⁶ cells/kg, or about 2.5×10⁶ cells/kg, or about 2.75×10⁶ cells/kg, or about 3×10⁶ cells/kg.

In some aspects, the therapeutic cells of the present disclosure can be administered in an amount that does not exceed about 5×10⁶ cells, or about 10×10⁶ cells, or about 15×10⁶ cells, or about 20×10⁶ cells, or about 25×10⁶ cells, or about 30×10⁶ cells, or about 35×10⁶ cells, or about 40×10⁶ cells, or about 45×10⁶ cells, or about 50×10⁶ cells, or about 55×10⁶ cells, or about 60×10⁶ cells, or about 65×10⁶ cells, or about 70×10⁶ cells, or about 75×10⁶ cells, or about 80×10⁶ cells, or about 85×10⁶ cells, or about 90×10⁶ cells, or about 95×10⁶ cells, or about 100×10⁶ cells, or about 105×10⁶ cells, or about 110×10⁶, or about 115×10⁶ cells, or about 120×10⁶ cells, or about 125×10⁶ cells, or about 130×10⁶ cells, or about 135×10⁶ cells, or about 140×10⁶ cells, or about 145×10⁶ cells, or about 150×10⁶ cells, or about 155×10⁶ cells, or about 160×10⁶ cells, or about 165×10⁶ cells, or about 170×10⁶ cells, or about 175×10⁶ cells, or about 180×10⁶ cells, or about 185×10⁶ cells, or about 190×10⁶ cells, or about 195×10⁶ cells, or about 200×10⁶ cells, or about 205×10⁶ cells, or about 210×10⁶, or about 215×10⁶ cells, or about 220×10⁶ cells, or about 225×10⁶ cells, or about 230×10⁶ cells, or about 235×10⁶ cells, or about 240×10⁶ cells, or about 245×10⁶ cells, or about 250×10⁶ cells, or about 300×10⁶, or about 350×10⁶, or about 400×10⁶, or about 450×10⁶, or about 500×10⁶, or about 600×10⁶, or about 700×10⁶.

The term “benefit” is used in the broadest sense and refers to any desirable effect and specifically includes clinical benefit as defined herein. Clinical benefit can be measured by assessing various endpoints, e.g., inhibition, to some extent, of disease progression, including slowing down and complete arrest; reduction in the number of disease episodes and/or symptoms; reduction in lesion size; inhibition (i.e., reduction, slowing down or complete stopping) of disease cell infiltration into adjacent peripheral organs and/or tissues; inhibition (i.e. reduction, slowing down or complete stopping) of disease spread; decrease of auto-immune response, which may, but does not have to, result in the regression or ablation of the disease lesion; relief, to some extent, of one or more symptoms associated with the disorder; increase in the length of disease-free presentation following treatment, e.g., progression-free survival; increased overall survival; higher response rate; and/or decreased mortality at a given point of time following treatment.

### Methods of Cell Production of the Present Disclosure

The present disclosure provides methods of producing the therapeutic cells and pluralities of therapeutic cells of the present disclosure.

Accordingly, the present disclosure provides methods of producing a plurality of cells expressing the anti-CD99 CAR polypeptides of the present disclosure, the method comprising: a) obtaining a plurality of cells from a subject; and b) introducing into the plurality of cells one or more nucleic acid molecules of the present disclosure, wherein the one or more nucleic acid molecules comprise one or more nucleic acid sequences encoding for an anti-CD99 CAR polypeptide of the present disclosure.

In applications wherein cells are taken from a subject and then eventually transplanted back into the subject therapeutically, these cells are said to be “autologous”. In aspects wherein cells are taken from a first subject and then eventually transplanted into a different, second subject, these cells are said to be “allogeneic”.

The term “introducing” is intended presenting to the cell the nucleic acid molecules in such a manner that the nucleic acid molecule gains access to the interior of the host cell. The methods of the present disclosure do not depend on a particular method for introducing a nucleic acid molecule into a host cell, only that the polynucleotide construct gains access to the interior of one cell of the host. Methods for introducing nucleic acid molecules (e.g. vectors) into bacteria, plants, fungi and animals are known in the art including, but not limited to, stable transformation methods, transient transformation methods, and virus-mediated methods. For example, lentiviral and retroviral transduction methods can be used. As would be appreciated by the skilled artisan, these methods can comprise the use of retronectin to enhance efficiency of transduction.

The methods of cell production described above can further comprise, before step (b), after step (b), or both before step (b) and after step (b), expanding the cells. Cell expansion can be accomplished using any cell expansion method known in the art.

The methods of cell production described above can further comprise, before step (b), after step (b), or both before step (b) and after step (b), culturing the cells. Cell culturing can be accomplished using any cell culturing method known in the art.

The manufacturing methods described above can further comprise, after step (b), enriching for cells that express an anti-CD99 CAR polypeptide of the present disclosure. The enrichment can be accomplished by contacting the plurality of cells with an affinity reagent that binds to the anti-CD99 CAR polypeptide of the present disclosure. In some aspects, the affinity reagent is an antibody that binds to the anti-CD99 CAR polypeptide of the present disclosure. In some aspects, following enrichment, at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 97%, or at least about 99%, or at least about 100% of the cells express the anti-CD99 CAR polypeptide of the present disclosure of the present disclosure.

The methods of producing a plurality of cells expressing the anti-CD99 CAR polypeptides can further comprise genetically modifying the cells such that the cells do not express CD99. This genetic modification can be performed either before or after the introduction of a nucleic acid comprising one more sequence encoding for the anti-CD99 CAR polypeptide of the present disclosure.

Accordingly, the present disclosure provides methods of producing a plurality of cells expressing the anti-CD99 CAR polypeptides of the present disclosure, the method comprising: a) obtaining a plurality of cells from a subject; b) genetically modifying the cells such that the cells do not express CD99; and c) introducing into the plurality of cells one or more nucleic acid molecules of the present disclosure, wherein the one or more nucleic acid molecules comprise one or more nucleic acid sequences encoding for an anti-CD99 CAR polypeptide of the present disclosure.

The cells can be genetically modified to not express CD99 using any suitable method known in the art. For example, the cells can be genetically modified using a CRISPR-based genetic modification system such that the cells do not express CD99 (see e.g. Dimitri et al. Engineering the next-generation of CAR T-cells with CRISPR-Cas9 gene editing. Mol Cancer 21, 78 (2022). https://doi.org/10.1186/12943-022-01559-2).

### Kits

The present disclosure provides kits comprising any of the compositions described, or any combination of the compositions described herein.

Accordingly, the present disclosure provides kits comprising: i) the anti-CD99 CAR polypeptides of the present disclosure; ii) the cells of the present disclosure; iii) the cell populations of the present disclosure; iv) the nucleic acid molecules of the present disclosure; or any combination thereof.

### General Methods and Definitions

### Methods of Gene Delivery and Cell Modification

As used herein, “vector” means a construct, which is capable of delivering, and, in some embodiments, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

One of skill in the art would be well-equipped to construct a vector through standard recombinant techniques (see, for example, Sambrook et al., 2001 and Ausubel et al, 1996, both incorporated herein by reference) for the expression of the antigen receptors of the present disclosure. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

### Viral Vectors

Viral vectors encoding an antigen receptor, a cytokine and/or an functional effector element may be provided in certain aspects of the methods of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor mediated-endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention are described below.

In some embodiments of the methods of the disclosure, introducing a nucleic acid sequence and/or a genomic editing construct into an immune cell ex vivo, in vivo, in vitro or in situ comprises a viral vector. In some embodiments, the viral vector is a non-integrating non-chromosomal vector. Exemplary non-integrating non-chromosomal vectors include, but are not limited to, adeno-associated virus (AAV), adenovirus, and herpes viruses. In some embodiments, the viral vector is an integrating chromosomal vector. Integrating chromosomal vectors include, but are not limited to, adeno-associated vectors (AAV), Lentiviruses, and gamma-retroviruses.

In some embodiments of the methods of the disclosure, introducing a nucleic acid sequence and/or a genomic editing construct into an immune cell ex vivo, in vivo, in vitro or in situ comprises a combination of vectors. Exemplary, non-limiting vector combinations include: viral and non-viral vectors, a plurality of non-viral vectors, or a plurality of viral vectors.

Exemplary but non-limiting vectors combinations include: a combination of a DNA-derived and an RNA-derived vector, a combination of an RNA and a reverse transcriptase, a combination of a transposon and a transposase, a combination of a non-viral vector and an endonuclease, and a combination of a viral vector and an endonuclease.

In some embodiments of the methods of the disclosure, genome modification comprising introducing a nucleic acid sequence and/or a genomic editing construct into an immune cell ex vivo, in vivo, in vitro or in situ stably integrates a nucleic acid sequence, transiently integrates a nucleic acid sequence, produces site-specific integration a nucleic acid sequence, or produces a biased integration of a nucleic acid sequence. In some embodiments, the nucleic acid sequence is a transgene.

In some embodiments of the methods of the disclosure, genome modification comprising introducing a nucleic acid sequence and/or a genomic editing construct into an immune cell ex vivo, in vivo, in vitro or in situ stably integrates a nucleic acid sequence. In some embodiments, the stable chromosomal integration can be a random integration, a site-specific integration, or a biased integration. In some embodiments, the site-specific integration can be non-assisted or assisted. In some embodiments, the assisted site-specific integration is co-delivered with a site-directed nuclease. In some embodiments, the site-directed nuclease comprises a transgene with 5′ and 3′ nucleotide sequence extensions that contain a percentage homology to upstream and downstream regions of the site of genomic integration. In some embodiments, the transgene with homologous nucleotide extensions enable genomic integration by homologous recombination, microhomology-mediated end joining, or nonhomologous end-joining. In some embodiments the site-specific integration occurs at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Potential genomic safe harbors include, but are not limited to, intronic sequences of the human albumin gene, the adeno-associated virus site 1 (AAVS1), a naturally occurring site of integration of AAV virus on chromosome 19, the site of the chemokine (C—C motif) receptor 5 (CCR5) gene and the site of the human ortholog of the mouse Rosa26 locus.

In some embodiments, the site-specific transgene integration occurs at a site that disrupts expression of a target gene. In some embodiments, disruption of target gene expression occurs by site-specific integration at introns, exons, promoters, genetic elements, enhancers, suppressors, start codons, stop codons, and response elements. In some embodiments, exemplary target genes targeted by site-specific integration include but are not limited to any immunosuppressive gene, and genes involved in allo-rejection.

In some embodiments, the site-specific transgene integration occurs at a site that results in enhanced expression of a target gene. In some embodiments, enhancement of target gene expression occurs by site-specific integration at introns, exons, promoters, genetic elements, enhancers, suppressors, start codons, stop codons, and response elements.

### Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters.

### Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated.

Alternatively, a replication origin of other extra-chromosomally replicating virus as described above or an autonomously replicating sequence (ARS) can be employed.

### Other Methods of Nucleic Acid Delivery

In addition to viral delivery of the nucleic acids encoding the antigen receptor, the following are additional methods of recombinant gene delivery to a given cell, (e.g. an NK cell) and are thus considered in the present disclosure.

Introduction of a nucleic acid molecule, such as DNA or RNA, into the immune cells of the current disclosure may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection); by electroporation; by calcium phosphate precipitation; by using DEAE-dextran followed by polyethylene glycol; by direct sonic loading; by liposome mediated transfection and receptor-mediated transfection; by lipid nanoparticle transfection; by microprojectile bombardment; by agitation with silicon carbide fibers; by Agrobacterium-mediated transformation; by desiccation/inhibition-mediated DNA uptake, and any combination of such methods. Through the application of techniques such as these, organelle(s), cell(s), tissue(s) or organism(s) may be stably or transiently transformed.

Generally, the gene transfer system can include a transposon-based or a viral-based integration system.

In some embodiments, the gene transfer system comprises a transposon system. DNA transposons can translocate via a non-replicative “cut-and-paste” mechanism. This mechanism requires recognition of the two inverse terminal repeats (ITRs) by a catalytic enzyme, i.e., transposase, which can cleave its target and consequently release the DNA transposon from its donor template. Upon excision, the DNA transposons may subsequently integrate into the acceptor DNA that is cleaved by the same transposase. In some of their natural configurations, DNA transposons are flanked by two ITRs and may contain a gene encoding a transposase that catalyzes transposition. As would be appreciated by the skilled artisan, transposon systems offer many advantages for nucleic acid integration, e.g., as compared to viral vectors. For example, transposons can carry larger cargos, which can be advantageous for delivering one or more of the CARs, functional effector elements, and/or cytokines disclosed herein, to an immune cell (e.g., an NK cell). Further, transposons may comprise, for example, CRISPR tools (e.g., along with cargo), and thereby allow multiplex engineering of a cell.

### Chimeric Antigen Receptors

A “chimeric antigen receptor” is also known as an artificial cell receptor, a chimeric cell receptor, or a chimeric immunoreceptor. As would be appreciated by the skilled artisan, Chimeric antigen receptors (CARs) are engineered receptors, which graft a selected specificity onto an immune effector cell. CARs typically have an extracellular domain (ectodomain), a transmembrane domain and an intracellular (endodomain) domain. In some embodiments, the ectodomain comprises an antigen-binding domain and a hinge domain, wherein the antigen-binding domain specifically binds to an antigen that is of particular interest in the treatment of a specific disease or disorder (e.g. an antigen that is located on particular cancer cells or an antigen located on an infected cell). In some embodiments, the antigen is a protein expressed on the surface of cells (e.g., on the surface of a cancer cell, or an infected cell).

As would be appreciated by the skilled artisan, CARs are available in various different “formats”, sometimes also referred to as different “generations” of CARs (see e.g. Hiltensperger M, Krackhardt A M. Current and future concepts for the generation and application of genetically engineered CAR-T and TCR-T cells. Front Immunol. 2023 Mar. 6; 14:1121030. doi: 10.3389/fimmu.2023.1121030. PMID: 36949949; PMCID: PMC10025359). Accordingly, the term engineered CAR as used herein encompasses any of the formats/generations known in the art.

In a non-limiting example, the CAR can be in the format of a universal CAR, such as those disclosed in PCT Publication No. WO/2012/082841 and U.S. Pat. Nos. 9,233,125 and 10,973,893. As would be appreciated by the skilled artisan, in universal CAR systems, the extracellular domain of the CAR specifically binds to a common “tag” molecule. Such CARs can be referred to as “anti-tag CARs”. These tag molecules can then be fused to antigen-targeting molecules (e.g. antibodies), such that the anti-tag CARs bind to a target cell of interest indirectly through the tag-fusion protein. In this way, subjects can be administered a single population of cells expressing an anti-tag CAR and then a variety of different tag-fusion proteins that can specifically directed the anti-tag CAR cells to target different cells.

### Engineered T Cell Receptors

As would be appreciated by the skilled artisan, a T cell Receptor (TCR) is a heterodimeric cell surface protein of the immunoglobulin super-family, which is associated with invariant proteins of the CD3 complex involved in mediating signal transduction. TCRs exist as αβ and γδ heterodimers, which are structurally similar but have quite distinct anatomical locations and probably functions. The extracellular portion of native heterodimeric PTCR consists of two polypeptide chains, each of which has a membrane-proximal constant domain, and a membrane-distal variable domain. Each of the constant and variable domains includes an intra-chain disulfide bond. The variable domains contain the highly polymorphic loops analogous to the complementarity determining regions (CDRs) of antibodies. As used herein, the term “engineered T cell receptor” refers to TCRs that have been designed to specifically bind to an antigen that is of particular interest in the treatment of a specific disease or disorder (e.g. an antigen that is located on particular cancer cells or an antigen located on an infected cell). In some embodiments, the antigen is a protein expressed on the surface of cells (e.g., on the surface of a cancer cell, or an infected cell). As would be appreciated by the skilled artisan, engineered TCRs are available in various different “formats” (see e.g. Hiltensperger M, Krackhardt A M. Current and future concepts for the generation and application of genetically engineered CAR-T and TCR-T cells. Front Immunol. 2023 Mar. 6; 14:1121030. doi: 10.3389/fimmu.2023.1121030. PMID: 36949949; PMCID: PMC10025359). Accordingly, the term engineered TCR as used herein encompasses any of the formats known in the art.

### Definitions

As would be appreciated by the skilled artisan, “CD99” refers to the protein known as cluster of differentiation 99, which is also referred to in the art as MIC2.

As used herein, the term “antigen” is a molecule capable of being bound by an antibody, T cell receptor, Chimeric Antigen Receptor and or engineered immune receptor. An antigen may generally be used to induce a humoral immune response and/or a cellular immune response leading to the production of B and/or T lymphocytes.

The terms “tumor-associated antigen,” “tumor antigen” and “cancer cell antigen” are used interchangeably herein. In each case, the terms refer to proteins, glycoproteins or carbohydrates that are specifically or preferentially expressed by cancer cells.

As used herein, the term “portion” when used in reference to a polypeptide or a peptide refers to a fragment of the polypeptide or peptide. In some embodiments, a “portion” of a polypeptide or peptide retains at least one function and/or activity of the full-length polypeptide or peptide from which it was derived. For example, in some embodiments, if a full-length polypeptide binds a given ligand, a portion of that full-length polypeptide also binds to the same ligand.

The terms “protein” and “polypeptide” are used interchangeably herein.

As known in the art, “nucleic acid molecule,” “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, the term refers to a cell that was isolated and subsequently introduced into a cell population or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from where it would be in natural cells, or is otherwise flanked by a different nucleic acid sequence than that found in nature. The term “exogenous” is used interchangeably with the term “heterologous”.

By “expression construct” or “expression cassette” is used to mean a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “gene,” “polynucleotide,” “coding region,” “sequence,” “nucleic acid sequence,” “segment,” “fragment,” or “transgene” that “encodes” a particular protein, is a section of a nucleic acid molecule that is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “cell” is herein used in its broadest sense in the art and refers to a living body that is a structural unit of tissue of a multicellular organism, is surrounded by a membrane structure that isolates it from the outside, has the capability of self-replicating, and has genetic information and a mechanism for expressing it. Cells used herein may be naturally-occurring cells or artificially modified cells (e.g., fusion cells, genetically modified cells, etc.).

“Antibody” as used herein refers to monoclonal or polyclonal antibodies. The term “monoclonal antibodies,” as used herein, refers to antibodies that are produced by a single clone of B-cells and bind to the same epitope. In contrast, “polyclonal antibodies” refer to a population of antibodies that are produced by different B-cells and bind to different epitopes of the same antigen. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CHL CH2 and CH3) regions, and each light chain contains one N-terminal variable (VL) region and one C-terminal constant (CL) region. The variable regions of each pair of light and heavy chains form the antigen binding site of an antibody. The VH and VL regions have a similar general structure, with each region comprising four framework regions, whose sequences are relatively conserved. The framework regions are connected by three complementarity determining regions (CDRs). The three CDRs, known as CDR1, CDR2, and CDR3, form the “hypervariable region” of an antibody, which is responsible for antigen binding.

The term “T cell” refers to T lymphocytes, and includes, but is not limited to, γ/δ T cells, α/β T cells, NK T cells, CD4⁺ T cells and CD8⁺ T cells. CD4⁺ T cells include THO, Tₕ1 and TH2 cells, as well as regulatory T cells (T_{reg}). There are at least three types of regulatory T cells: CD4⁺ CD25⁺ T_{reg}, CD25 T_{H}3 T_{reg}, and CD25 T_{R} 1 T_{reg}. “Cytotoxic T cell” refers to a T cell that can kill another cell. The majority of cytotoxic T cells are CD8⁺ MHC class I-restricted T cells, however some cytotoxic T cells are CD4⁺. In some embodiments, the T cell of the present disclosure is CD4⁺ or CD8+.

The activation state of a T cell defines whether the T cell is “resting” (i.e., in the Go phase of the cell cycle) or “activated” to proliferate after an appropriate stimulus such as the recognition of its specific antigen, or by stimulation with OKT3 antibody, PHA or PMA, etc. The “phenotype” of the T cell (e.g., naive, central memory, effector memory, lytic effectors, help effectors (THI and TH2 cells), and regulatory effectors), describes the function the cell exerts when activated. A healthy donor has T cells of each of these phenotypes, and which are predominately in the resting state. A naive T cell will proliferate upon activation, and then differentiate into a memory T cell or an effector T cell. It can then assume the resting state again, until it gets activated the next time, to exert its new function and may change its phenotype again. An effector T cell will divide upon activation and antigen-specific effector function.

“Natural killer T cells” (NKT cells), not to be confused with natural killer cells of the innate immune system, bridge the adaptive immune system with the innate immune system. Unlike conventional T cells that recognize peptide antigens presented by major histocompatibility complex (WIC) molecules, NKT cells recognize glycolipid antigen presented by a molecule called CD1d. Once activated, these cells can perform functions ascribed to both Th and Tc cells (i.e., cytokine production and release of cytolytic/cell killing molecules). They are also able to recognize and eliminate some tumor cells and cells infected with herpes viruses.

“Natural killer cells” (“NK cells”) are a type of cytotoxic lymphocyte of the innate immune system. In some instances, NK cells provide a first line defense against viral infections and/or tumor formation. NK cells can detect MHC presented on infected or cancerous cells, triggering cytokine release, and subsequently induce lysis and apoptosis. NK cells can further detect stressed cells in the absence of antibodies and/or MHC, thereby allowing a rapid immune response.

The term “culturing” refers to the in vitro maintenance, differentiation, and/or propagation of cells in suitable media. By “enriched” is meant a composition comprising cells present in a greater percentage of total cells than is found in the tissues where they are present in an organism.

As used throughout the disclosure, identity between two sequences may be determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. In some embodiments, the sequence identify is determined over the entire length of a sequence. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

### Exemplary Embodiments

Embodiment 1. An anti-CD99 single chain variable fragment (scFv) comprising a CDHR1 having the amino acid sequence of SEQ ID NO: 1, a CDRH2 having the amino acid sequence of SEQ ID NO: 2, a CDRH3 having the amino acid sequence of SEQ ID NO: 3, a CDRL1 having the amino acid sequence of SEQ ID NO: 4, a CDRL2 having the amino acid sequence of SEQ ID NO: 5, a CDHL3 having the amino acid sequence of SEQ ID NO: 6.

Embodiment 2. The anti-CD99 scFv of embodiment 1, wherein the anti-CD99 scFv comprises a VH domain comprising the amino acid sequence of SEQ ID NO: 7 and a VL domain comprising the amino acid sequence of SEQ ID NO: 9.

Embodiment 3. The anti-CD99 scFv of embodiment 2, wherein the anti-CD99 scFv comprises a linker domain connecting the VH domain and the VL domain, wherein the linker domain comprises the amino acid sequence of SEQ ID NO: 11.

Embodiment 4. An anti-CD99 single chain variable fragment (scFv) comprising the amino acid sequence of SEQ ID NO: 12.

Embodiment 5. An anti-CD99 CAR polypeptide comprising from N-terminus to C-terminus, an anti-CD99 antigen binding domain, a transmembrane domain, a costimulatory domain, and an activation domain.

Embodiment 6. The anti-CD99 CAR polypeptide of embodiment 5, wherein the anti-CD99 CAR polypeptide further comprises a hinge domain located between the anti-CD99 antigen binding domain and the transmembrane domain.

Embodiment 7. The anti-CD99 CAR polypeptide of any one of the preceding embodiments, wherein the anti-CD99 CAR polypeptide further comprises a signal peptide at the N-terminus.

Embodiment 8. The anti-CD99 CAR polypeptide of any one of the preceding embodiments, wherein the anti-CD99 antigen binding domain comprises the anti-CD99 scFv of any one of embodiments 1-4.

Embodiment 9. The anti-CD99 CAR polypeptide of any one of the preceding embodiments, wherein the transmembrane domain comprises a CD8 transmembrane domain.

Embodiment 10. The anti-CD99 CAR polypeptide of any one of the preceding embodiments, wherein the CD8 transmembrane domain comprises the amino acid sequence of SEQ ID NO: 20.

Embodiment 11. The anti-CD99 CAR polypeptide of any one of the preceding embodiments, wherein the transmembrane domain comprises a CD28 transmembrane domain.

Embodiment 12. The anti-CD99 CAR polypeptide of any one of the preceding embodiments, wherein the CD28 transmembrane domain comprises the amino acid sequence of SEQ ID NO: 18.

Embodiment 13. The anti-CD99 CAR polypeptide of any one of the preceding embodiments, wherein the costimulatory domain comprises at least one 4-1BB costimulatory polypeptide.

Embodiment 14. The anti-CD99 CAR polypeptide of any one of the preceding embodiments, wherein the at least one 4-1BB costimulatory polypeptide comprises the amino acid sequence of SEQ ID NO: 24.

Embodiment 15. The anti-CD99 CAR polypeptide of any one of the preceding embodiments, wherein the costimulatory domain comprises at least one CD28 costimulatory polypeptide.

Embodiment 16. The anti-CD99 CAR polypeptide of any one of the preceding embodiments, wherein the at least one CD28 costimulatory polypeptide comprises the amino acid sequence of SEQ ID NO: 22.

Embodiment 17. The anti-CD99 CAR polypeptide of any one of the preceding embodiments, wherein the activation domain comprises a CD3ζ activation polypeptide.

Embodiment 18. The anti-CD99 CAR polypeptide of any one of the preceding embodiments, wherein the CD3ζ activation polypeptide comprises the amino acid sequence of SEQ ID NO: 26.

Embodiment 19. The anti-CD99 CAR polypeptide of any one of the preceding embodiments, wherein the signal peptide comprises a CD8 signal peptide.

Embodiment 20. The anti-CD99 CAR polypeptide of any one of the preceding embodiments, wherein the CD8 signal peptide comprises the amino acid sequence of SEQ ID NO: 28.

Embodiment 21. The anti-CD99 CAR polypeptide of any one of the preceding embodiments, wherein the signal peptide comprises a GMCSF signal peptide.

Embodiment 22. The anti-CD99 CAR polypeptide of any one of the preceding embodiments, wherein the GMCSF signal peptide comprises the amino acid sequence of SEQ ID NO: 30.

Embodiment 23. The anti-CD99 CAR polypeptide of any one of the preceding embodiments, wherein the hinge domain comprises a CD8 hinge domain.

Embodiment 24. The anti-CD99 CAR polypeptide of any one of the preceding embodiments, wherein the CD8 hinge domain comprises the amino acid sequence of SEQ ID NO: 16.

Embodiment 25. The anti-CD99 CAR polypeptide of any one of the preceding embodiments, wherein the hinge domain comprises a CD28 hinge domain.

Embodiment 26. The anti-CD99 CAR polypeptide of any one of the preceding embodiments, wherein the CD28 hinge domain comprises the amino acid sequence of SEQ ID NO: 14.

Embodiment 27. The anti-CD99 CAR polypeptide of any one of the preceding embodiments, wherein the anti-CD99 CAR polypeptide comprises, from N-terminus to C-terminus, a signal peptide comprising a CD8 signal peptide, an anti-CD99 antigen binding domain comprising an anti-CD99 scFv, a hinge domain comprising a CD8 hinge polypeptide, a transmembrane domain comprising a CD8 transmembrane polypeptide, a costimulatory domain comprising a 4-1BB costimulatory polypeptide, and an activation domain comprising CD3ζ activation polypeptide.

Embodiment 28. The anti-CD99 CAR polypeptide of any one of the preceding embodiments, wherein the anti-CD99 CAR polypeptide comprises the amino acid sequence of SEQ ID NO: 32.

Embodiment 29. The anti-CD99 CAR polypeptide of any one of the preceding embodiments, wherein the anti-CD99 CAR polypeptide comprises, from N-terminus to C-terminus, a signal peptide comprising a CD8 signal peptide, an anti-CD99 antigen binding domain comprising an anti-CD99 scFv, a hinge domain comprising a CD28 hinge polypeptide, a transmembrane domain comprising a CD28 transmembrane polypeptide, a costimulatory domain comprising a CD28 costimulatory polypeptide, and an activation domain comprising CD3ζ activation polypeptide.

Embodiment 30. The anti-CD99 CAR polypeptide of any one of the preceding embodiments, wherein the anti-CD99 CAR polypeptide comprises the amino acid sequence of SEQ ID NO: 34.

Embodiment 31. A nucleic acid molecule comprising one or more nucleic acid sequences encoding for the anti-CD99 scFv of any one of embodiments 1-4.

Embodiment 32. A nucleic acid molecule comprising one or more nucleic acid sequences encoding for the anti-CD99 CAR polypeptide of any one of embodiments 5-30.

Embodiment 33. The nucleic acid molecule of any one of embodiments 31-32, wherein the nucleic acid molecule comprises the nucleic acid sequence of SEQ ID NO: 13.

Embodiment 34. The nucleic acid molecule of any one of embodiments 31-33, wherein the nucleic acid molecule comprises the nucleic acid sequence of SEQ ID NO: 33.

Embodiment 35. The nucleic acid molecule of any one of embodiments 31-34, wherein the nucleic acid molecule comprises the nucleic acid sequence of SEQ ID NO: 35.

Embodiment 36. A vector comprising the nucleic acid molecule of any one of embodiments 31-35.

Embodiment 37. The vector of embodiment 36, wherein the vector is a viral vector.

Embodiment 38. The vector of embodiment 37, wherein the viral vector is an AAV vector or a lentiviral vector.

Embodiment 39. A cell expressing the anti-CD99 CAR polypeptide of any one of embodiments 5-30.

Embodiment 40. The cell of embodiment 39, wherein the cell does not express CD99.

Embodiment 41. The cell of any one of embodiments 39-40, wherein the cell is an immune cell.

Embodiment 42. The cell of embodiment 41, wherein the immune cell is a T cell, NK cell, NK-like cell, NKT cell, or cytokine induced killer (CIK) cell.

Embodiment 43. The cell of embodiment 42, wherein the immune cell is a T cell.

Embodiment 44. A population of the cells of any one of embodiments 39-43.

Embodiment 45a. A method of treating cancer in a subject, the method comprising administering to the subject one or more amounts of the cell population of embodiment 44.

Embodiment 45a. A method of preventing cancer metastasis in a subject, the method comprising administering to the subject one or more amounts of the cell population of embodiment 44.

Embodiment 46. The method of embodiment 45, the method further comprising administering to the subject at least one additional therapy.

Embodiment 47. The method of embodiment 46, wherein the at least one additional therapy comprises radiation therapy.

Embodiment 48. The method of any one of embodiments 45-47, wherein the at least one additional therapy comprises chemotherapy.

Embodiment 49. The method of any one of embodiments 45-48, wherein the cancer is characterized by expression of CD99 in the cancerous cells.

Embodiment 50. The method of any one of embodiments 45-49, wherein the cancer is diffuse intrinsic pontine glioma.

Embodiment 51. The method of any one of embodiments 45-50, wherein the cancer is acute myeloid leukemia.

Embodiment 52. The method of any one of embodiments 45-51, wherein the cancer is Ewing Sarcoma.

Embodiment 53. The method of any one of embodiments 45-52, wherein the cancer is a large granular lymphocyte leukemia.

Embodiment 54. The method of any one of embodiments 45-53, wherein the cancer is a chronic myeloid leukemia

Embodiment 55. The method of any one of embodiments 45-54, wherein the cancer is a glioblastoma.

Embodiment 56. The method of any one of embodiments 45-55, wherein the cancer is a glioma.

Embodiment 57. The method of any one of embodiments 45-56, wherein the cancer is an ependymoma.

Embodiment 58. The method of any one of embodiments 45-57, wherein the cancer is an atypical teratoid/rhabdoid tumor.

Embodiment 59. The method of any one of embodiments 45-58, wherein the cancer is a neuroblastoma.

Embodiment 60. The method of any one of embodiments 45-59, wherein the cancer is glioblastoma multiforme.

Embodiment 61. The method of any one of embodiments 45-60, wherein the cancer is recurrent cancer.

Embodiment 62. The method of any one of embodiments 45-61, wherein the cancer is refractory cancer.

Embodiment 63. The method of any one of embodiments 45-62, wherein the cancer has metastasized.

Embodiment 64. The method of any one of embodiments 45-63, wherein the subject has been previously administered at least one previous therapy.

Embodiment 65. The method of embodiment 45-64, wherein the at least one previous therapy comprises radiation therapy.

Embodiment 66. The method of any one of embodiments 45-65, wherein the at least one previous therapy comprises chemotherapy.

Embodiment 67. The method of any one of embodiments 45-66, wherein the at least one previous therapy comprises surgery.

Embodiment 68. The method of any one of embodiments 45-67, wherein the surgery comprises amputation of one or more body parts.

### EXAMPLES

### Example 1. Expression of CD99 is Elevated in H3K27M-DIPG Patient Tumors and Cell Lines

The following experimental example tests the role of the H3K27M mutant in diffuse intrinsic pontine glioma (DIPG) tumorigenesis. Use of integrated RNA-seq and Chip-seq data analysis in the isogenic models of H3K27M-mutant modified cell lines revealed genes that play a role in promoting DIPG cell proliferation. Applicants observed high levels of CD99 on the surface of multiple DIPG tumor cells with little to no expression on normal pons.

CRISPR-Cas9 mediated knockout of the H3K27M mutant in DIPG cells resulted in substantial decrease in CD99 expression. Furthermore, knockdown of CD99 or inhibition of CD99 using an anti-CD99 antibody impaired DIPG cell growth.

Without wishing to be bound by theory, these results indicate that overexpression of CD99 in H3K27M-DIPG patient samples and on H3K27M mutant cell lines is a specific consequence of the H3K27M mutation.

### Example 2. Functionality of CD99(10D1)-BBz CAR-T Cells Against DIPG In Vitro

The following experimental example tests the functionality of novel anti-CD99 CAR-T cells. Applicants generated a chimeric antigen receptor (CAR) incorporating a single chain variable fragment (scFv) having the amino acid sequence of SEQ ID NO: 12, which binds the extracellular domains of CD99. This scFv was engineered into a CAR with a 4-1BB co-stimulatory domain and the signaling domain of the CD3ζ chain to generate the CD99(10D1)-BBz CAR (SEQ ID NO: 32). Applicants found the transduction efficiency to be 84% using a G4S antibody, as measured by flow cytometry.

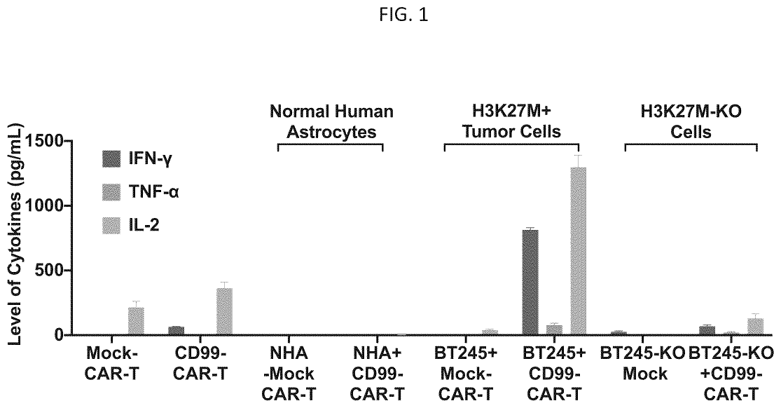

Applicants tested the effect of CD99(10D1)-BBz CAR-T cells on normal human astrocytes (NHA). Antigen dependent cytokine production was seen only with anti-CD99 CAR-T cells when co-cultured with H3K27M-DIPG cells and not with wild-type NHA cells or with CD99-negative-DIPG cells (CRISPR/Cas9 knockout of H3K27M). Furthermore, DIPG cells co-cultured with mock T cells or T cells transduced with a mock CAR (CD19 CAR) showed no cytokine production or tumor lysis (FIG. 1).

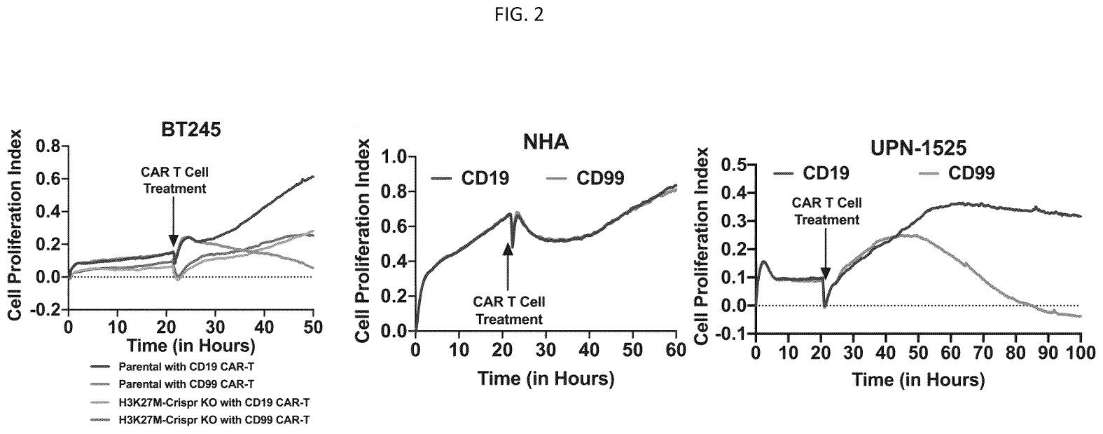

When co-incubated, CD99(10D1)-BBz CAR-T cells, but not anti-CD19 CAR-T cells, decreased the viability of H3K27M-DIPG-GFP cells. Additionally, CD99(10D1)-BBz CAR-T cells co-incubated with H3K27M-DIPG cells and primary H3K27M-DIPG patient samples (UPN-1525) in a 1:1 ratio resulted in significantly increased tumor cell lysis and decreased cell proliferation (FIG. 2). In contrast, CD99(10D1)-BBz CAR-T cells co-incubated with either NHA cells or CD99-negative cells (CRISPR/Cas9 knockout of H3K27M) resulted in no change in cell proliferation (FIG. 2). Furthermore, anti-CD19 CAR-T cells did not demonstrate cytokine production or lysis of H3K27M-DIPG cells (FIG. 1, FIG. 2). Applicants co-cultured the CD99(10D1)-BBz CAR-T cells with a H3K27M-DIPG short-term primary culture derived directly from a tumor biopsy (UPN-1525), resulting in abrogated tumor cell growth (FIG. 2).

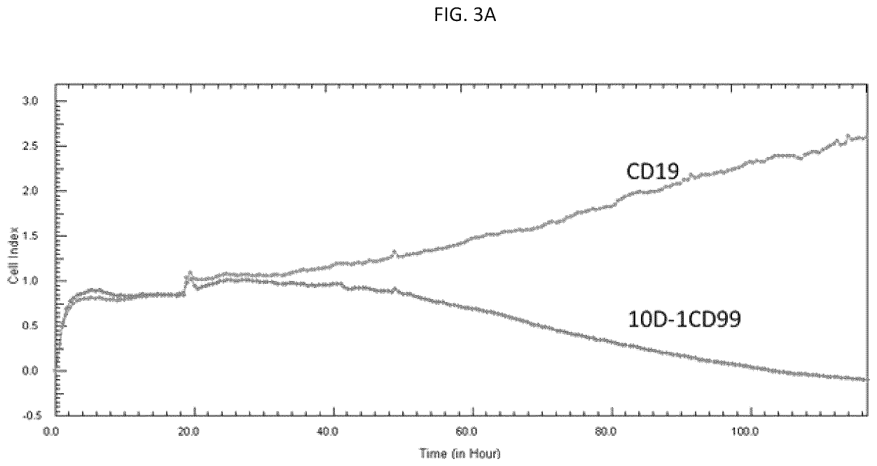

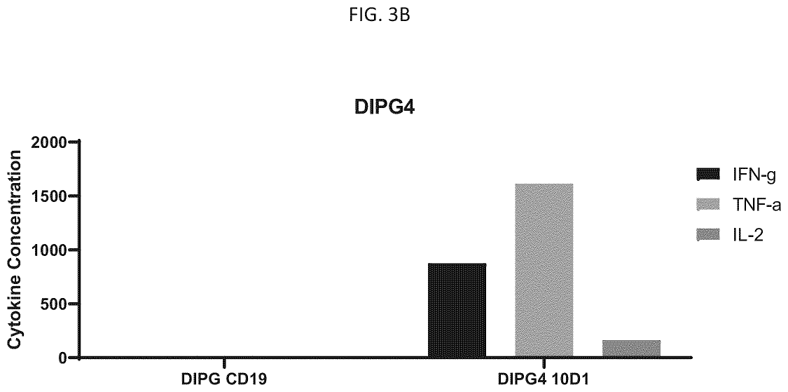

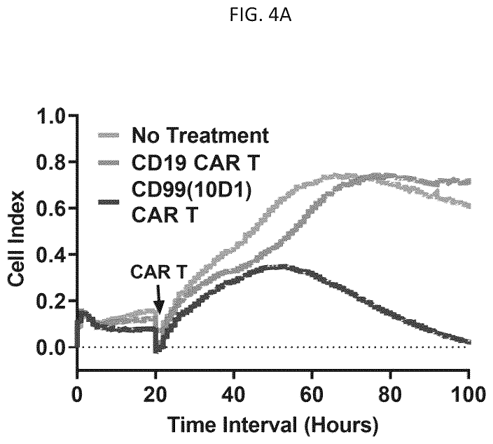

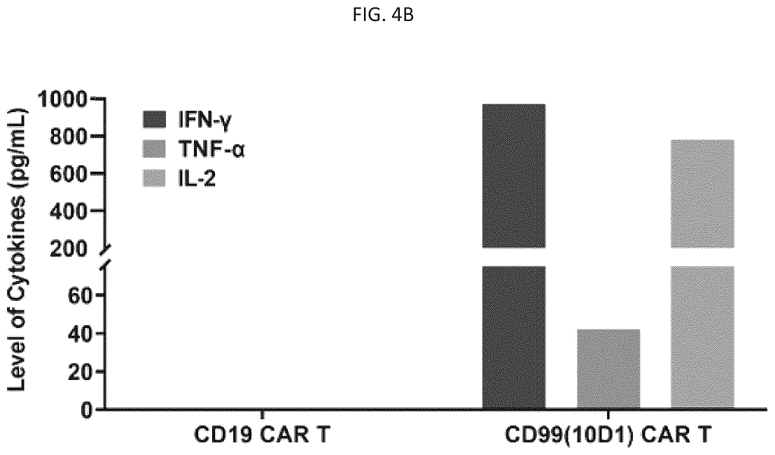

Applicants tested the functionality of CD99(10D1)-BBz CAR-T cells against DIPG4 tumor cells and primary patient-derived DIPG tumor cells (MAF-002). DIPG4 tumor cells were treated with CD99(10D1)-BBz CAR-T cells at a 1:1 (tumor cell: CAR-T cell) ratio and changes in growth were measured using XCELLigence real-time measurements that showed complete tumor lysis (FIG. 3A). Cytokine production was also increased when DIPG4 cells were treated with CD99(10D1)-BBz CAR-T cells at a 1:1 ratio, as compared to DIPG4 cells treated with anti-CD19 CAR-T cells (FIG. 3B). Primary patient-derived DIPG tumor cells (MAF-002) were treated with CD99(10D1)-BBz CAR-T cells and showed effective tumor lysis at a 1:1 (tumor cell: CAR-T cell) ratio, as measured using XCELLigence real-time measurements (FIG. 4A). Cytokine production was increased when MAF-002 cells were treated with CD99(10D1)-BBz CAR-T cells at a 1:1 ratio, as compared to MAF-002 cells treated with CD19 CAR-T cells (FIG. 4B).

Taken together, these results indicate that CD99(10D1)-BBz CAR-T cells are specific in targeting tumor cells highly expressing CD99 and CD99(10D1)-BBz CAR-T cells are a potent therapy for DIPG tumors.

### Example 3. CD99(10D1)-BBz CAR-T Cell Functionality Against DIPG Tumors In Vivo

The following experimental example tests the functionality of CD99(10D1)-BBz CAR-T cells against DIPG tumors in vivo. Applicants found that CD99(10D1)-BBz CAR-T cells lead to initial complete clearance of tumor burden (monitored by in vivo bioluminescence imaging), prolonged survival, and weight gain post treatment, relative to treatment with mock CD19 CAR-T cells.

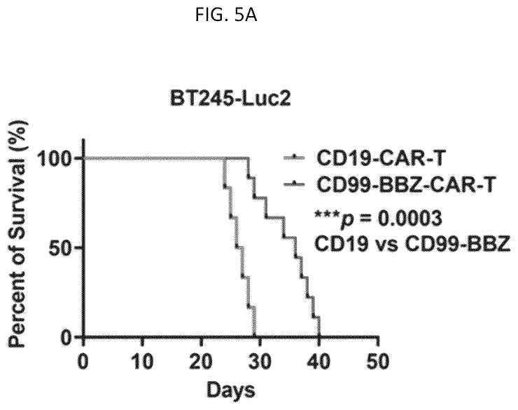

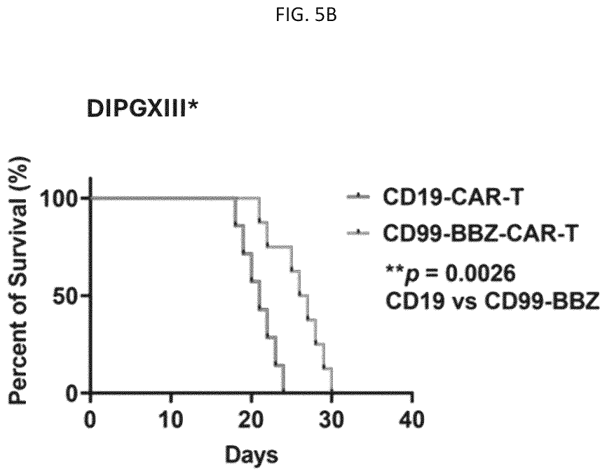

Applicants tested the functionality of CD99(10D1)-BBz CAR-T cells against mice bearing DIPG tumors in their pons. Two DIPG tumor bearing mouse models, BT245-luciferase and DIPG13-luciferase were treated with a single dose infusion of either anti-CD19 CAR-T cells or CD99(10D1)-BBz CAR-T cells at a dose of 20 million cells. CD99(10D1)-BBz CAR-T cells cleared the tumor by day 9 post treatment, whereas anti-CD19 CAR-T cells were ineffective against tumor clearance. Kaplan Meier's survival analysis of the two DIPG-pons bearing mouse models post infusion indicates that the CD99(10D1)-BBz CAR-T treatment resulted in a significant increase in animal survival compared to anti-CD19 CAR-T treatment in both tumor models (FIGS. 5A-5B).

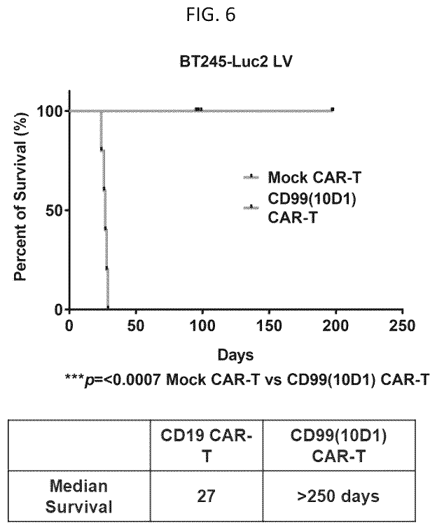

Applicants tested intrathecal delivery of CD99(10D1)-BBz CAR-T cells in both DIPG tumor mouse models. NSG (immunodeficient) mice were implanted with BT245 DIPG-Luciferase tumor cells in the pons, and after tumor establishment, a single dose (5×10⁶) of either CD99(10D1)-BBz CAR-T cells or anti-CD19 CAR-T cells was delivered to the lateral ventricle. CD99(10D1)-BBz CAR-T cells effectively cleared the DIPG tumor by day 6 post treatment, whereas anti-CD19 CAR-T cells were ineffective at clearing the DIPG tumor. The Kaplan-Meier survival analysis showed 100% survival of CD99(10D1)-BBz CAR-T cell treated mice after 250 days post treatment, whereas anti-CD19 CAR-T cell treated mice survived only 27 days post treatment (FIG. 6).

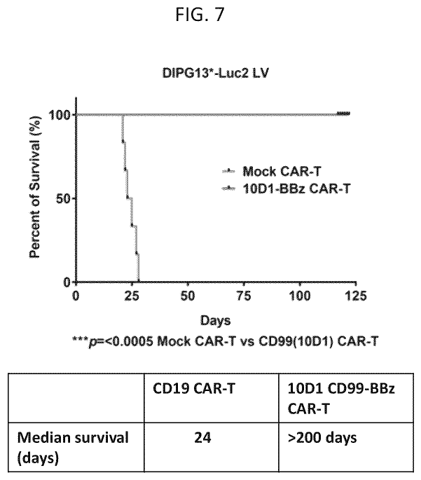

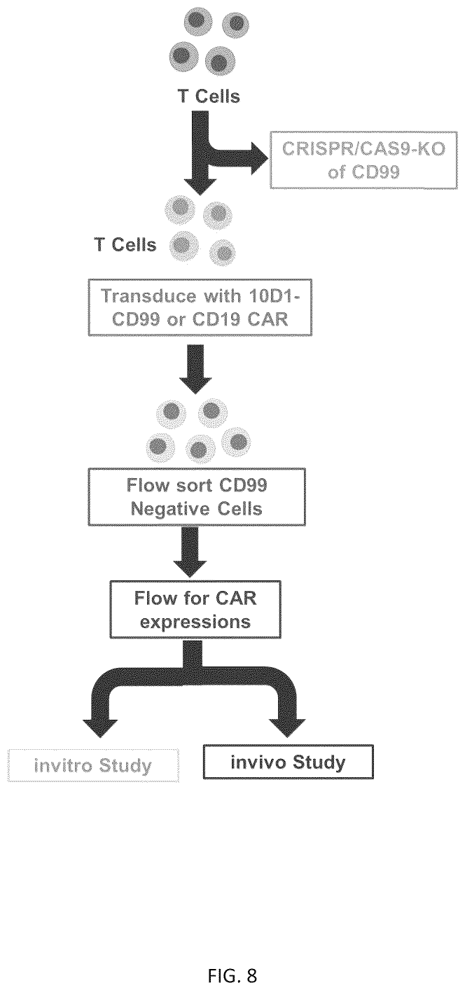

Similarly, applicants tested loco-regional delivery of CD99(10D1)-BBz CAR-T cells in NSG (immunodeficient) mice that were implanted with DIPG13-Luciferase tumor cells in the pons. The Kaplan-Meier survival analysis showed 100% survival of CD99(10D1)-BBz CAR-T cell treated mice after 200 days post treatment, whereas anti-CD19 CAR-T cell treated mice survived only 24 days post treatment (FIG. 7). Taken together, these results indicate that CD99(10D1)-BBz CAR-T cells that are delivered intrathecally are effective against DIPG tumors in vivo.

However, tumor reoccurrence was observed in mice over time. CAR-treated relapsed tumor tissues were investigated by immunohistochemistry (IHC) analysis and the presence of increased CD8⁺ cells at the tumor site were found, indicating that poor persistence of T cells is not the cause for tumor reoccurrence. Additionally, no expression of CD11b, a myeloid cell expression marker, was found indicating the myeloid infiltration was minimal. Furthermore, a second dose of CD99(10D1)-BBz CAR-T cells was given by tail-vein to a mouse that showed tumor reoccurrence after the initial CD99(10D1)-BBz CAR-T treatment and no change in tumor volume was noted, indicating that the administration of a second dose of the CAR-T cells does not improve animal survival. Taken together, these results indicate that the observed tumor reoccurrence is due to antigen modulation.

### Example 4. Radiation Prior to CD99(10D1)-BBz CAR-T Cell Therapy Acts Synergistically with CAR-T Therapy

The following experimental example tests the effect radiation, in combination with anti-CD99 CAR-T cell therapy has on DIPG tumor cells.

Applicants treated H3K27M-mutant DIPG cell lines (BT245 and DIPG13) with fractional radiation using clinically relevant Cesium-137. Cells were exposed to 4 Gy radiation using a research irradiator (JL Shepherd Model 81-14R) for 3 consecutive days totaling 12 Gy. At 24 hours post final radiation dose, flow cytometric analysis showed that radiation increased CD99 cell surface expression in BT245 and DIPG13 cells by 25% and 43%, respectively, relative to non-irradiated cells. To further test whether this observation is recapitulated in vivo, applicants implanted DIPG cells in the mouse pons and irradiated established tumors with a single dose of 8 Gy. The results showed a decrease in tumor volume initially after radiation but progressed within a period of 7-10 days.

The ability of focal radiation to synergize with the presently disclosed novel CD99 CAR-T cell therapy was tested in DIPG mouse xenografts. Immunodeficient mice (NSG) were inoculated with Luciferase (Luc)-expressing DIPG cells in their pons via an osmotic pump. After tumor establishment, mice were subjected to focal radiation using an image guided XRAD SmART image-guided irradiator. Each mouse received a single dose of 3 Gy to the tumor bed. Seven days post irradiation, the mice received either CD99(10D1)-BBz CAR-T treatment, anti-CD19 CAR-T treatment (negative control) or no CAR-T treatment by tail vein injections. Animals were monitored for changes in tumor volume by IVIS imaging.

Animals receiving radiation along with CD99 CAR-T treatment showed complete regression of tumor, prolonged survival, and no tumor reoccurrence. In contrast, mice treated with either CD19 control or mice receiving only radiation treatment (without CAR-T treatment), demonstrated progressive tumor growth and succumbed to their tumors.

Taken together, these data show that radiation prior to CD99(10D1)-BBz CAR-T cell therapy acts synergistically with CAR-T therapy, increasing the potency and efficacy of the therapy long-term.

### Example 5. CRISPR/Cas9 Mediated Knockout of CD99 from Donor T Cells Followed by Transduction of CD99 CAR-T Cells for DIPG CAR-T Therapy

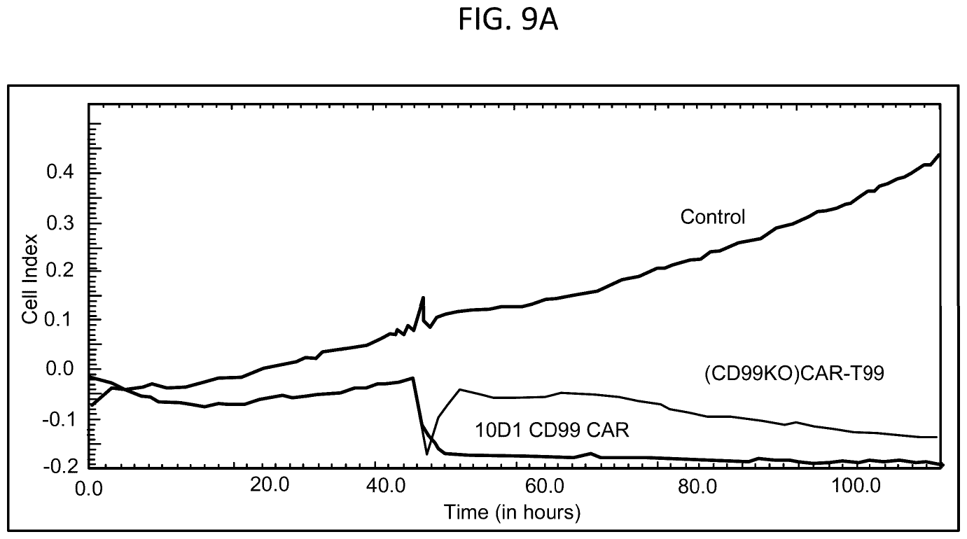

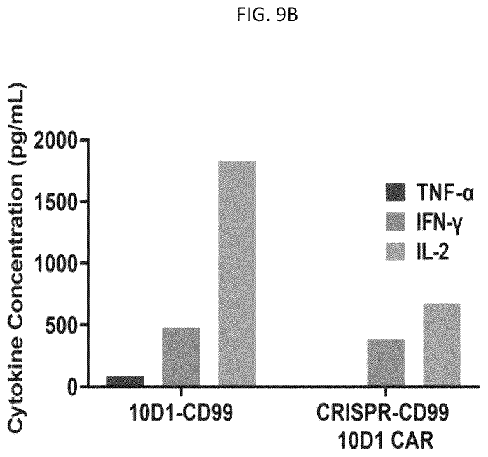

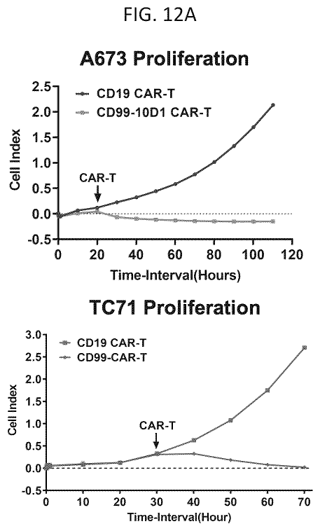

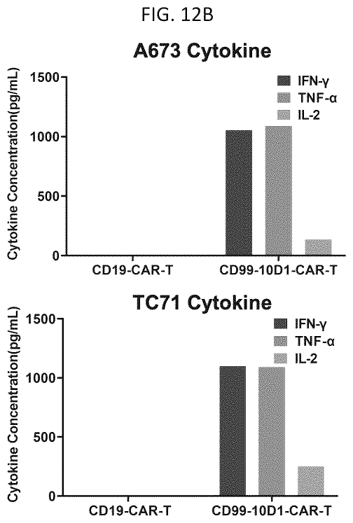

The following experimental example tests the functionality of CRISPR/Cas mediated knockout of CD99 from human donor T cells, followed by the transduction of CD99(10D1)-BBz CAR-T cells. CRISPR/Cas9 was utilized to generate a knockout of CD99 in human donor T cells. Pure CD99-negative cells (CD99KO) were then isolated for CAR-Transduction (FIG. 12). Applicants found that CD99KO CAR-T cells rescue T cells from CD99-derived fratricide (self-killing). DIPG4 tumor cells were treated with unedited CD99(10D1)-BBz CAR-T cells, edited CD99KO CD99(10D1)-BBz CAR-T cells, or CD19 CAR-T cells and it was found that DIPG4 tumor cells treated with either unedited CD99(10D1)-BBz CAR-T cells and edited CD99KO CD99(10D1)-BBz CAR-T cells show a decrease in cell proliferation, as compared to DIPG4 tumor cells treated with CD19 CAR-T cells (FIG. 9A). Furthermore, cytokine production was increased 24 hours post treatment of DIPG tumor cells with either unedited CD99(10D1)-BBz CAR-T cells or edited CD99KO CD99(10D1)-BBz CAR-T cells (FIG. 9B).

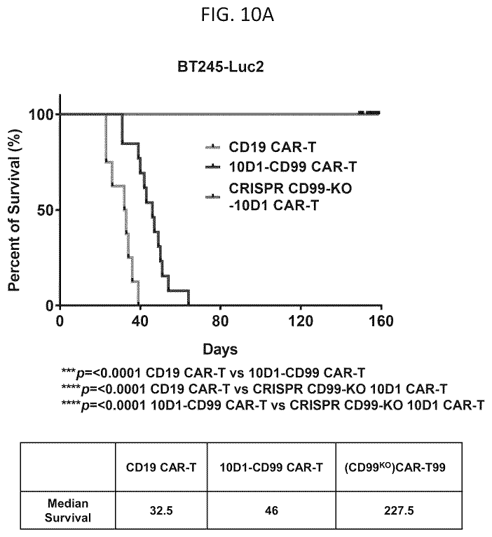

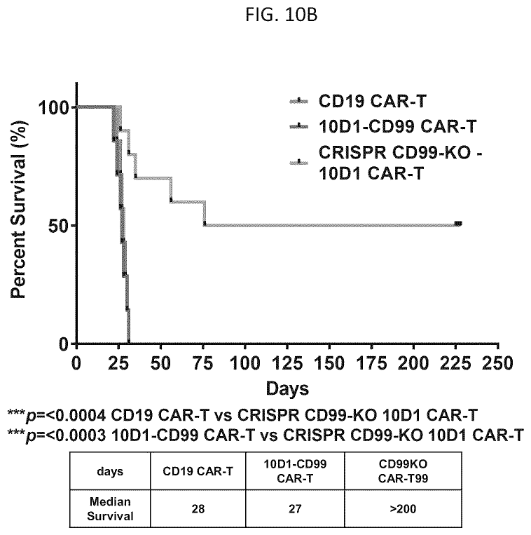

Applicants tested the functionality of unedited CD99(10D1)-BBz CAR-T cells and edited CD99KO CAR-T cells against mice bearing DIPG tumors in their pons. Two DIPG tumor bearing mouse models, BT245-GFP-Luciferase and DIPG13-GFP-Luciferase were treated with a single dose infusion, delivered systemically, of anti-CD19 CAR-T cells, edited CD99KO CD99(10D1)-BBz CAR-T cells, or unedited CD99(10D1)-BBz CAR-T cells at a dose of 20 million cells (anti-CD19 CAR-T cells and CD99(10D1)-BBz CAR-T cells) or 15 million cells (CD99KO CD99(10D1)-BBz CAR-T cells). Unedited CD99(10D1)-BBz CAR-T cells partially cleared the tumor by day 9 post treatment, edited CD99KO CD99(10D1)-BBz CAR-T cells completely cleared the tumor by day 9 post treatment, whereas anti-CD19 CAR-T cells were ineffective for tumor clearance. Kaplan Meier's survival analysis of the BT245-GFP-Luciferase DIPG-pons bearing mouse model post treatment indicates that the edited CD99KO CD99(10D1)-BBz CAR-T treatment resulting in animal survival 227.5 days post treatment, unedited CD99(10D1)-BBz CAR-T treatment resulted in animal survival of 46 days post treatment, and CD19 CAR-T treatment resulted in animal survival 32.5 days post treatment (FIG. 10A). Kaplan Meier's survival analysis of the DIPG13-GFP-Luciferase DIPG-pons bearing mouse model post treatment indicates that the edited CD99KO CAR-T treatment resulting in animal survival over 200 days post treatment, unedited CD99(10D1)-BBz CAR-T treatment resulted in animal survival of 27 days post treatment, and CD19 CAR-T treatment resulted in animal survival 28 days post treatment (FIG. 10B).

Taken together, these results show that edited CD99KO CD99(10D1)-BBz CAR-T cells, delivered systemically, are effective against DIPG tumors in vivo.

### Example 6. Functionality of CD99(10D1)-BBz CAR-T Cells Against Ewing Sarcoma

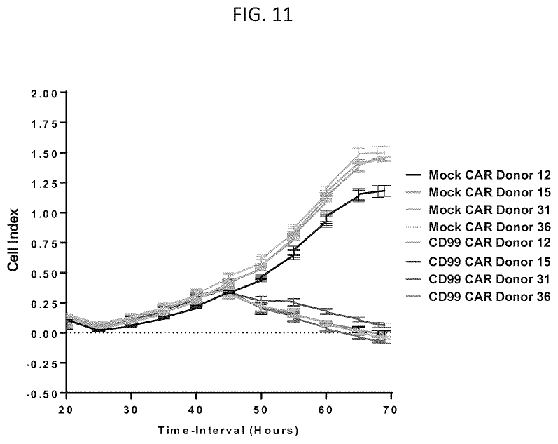

The following experimental example tests the functionality of CD99(10D1)-BBz CAR-T cells against Ewing Sarcoma. Applicants found that CD99 was highly expressed in Ewing Sarcoma cell lines and that CD99(10D1)-BBz CAR-T cells were effective in killing tumor cells from different human Ewing Sarcoma donor cells (FIG. 11).

Applicants tested the functionality of CD99(10D1)-BBz CAR-T cells against Ewing Sarcoma tumor cell lines (A673 and TC71). Ewing Sarcoma tumor cells were treated with CD99(10D1)-BBz CAR-T cells at a 1:1 (tumor cell: CAR-T cell) ratio and changes in growth were measured using XCELLigence real-time measurements that showed complete tumor lysis in both cell lines treated with CD99(10D1)-BBz CAR-T cells (FIG. 12A). Cytokine production was also increased when Ewing Sarcoma cells were treated with CD99(10D1)-BBz CAR-T cells at a 1:1 ratio, as compared to Ewing Sarcoma cells treated with CD19 CAR-T cells (FIG. 12B).

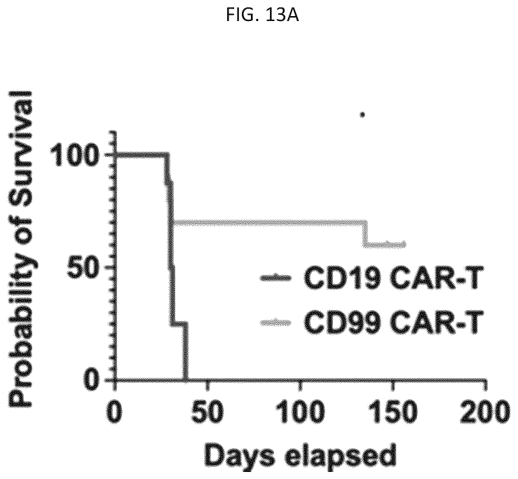

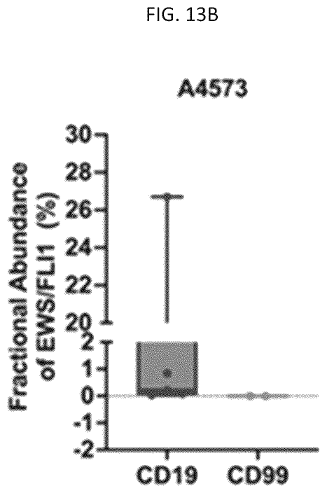

Applicants found that CD99(10D1)-BBz CAR-T cell treatment on A4573 orthotopic implantation amputation (OIA) mouse xenografts lead to initial complete clearance of tumor burden (monitored by IVIS imaging) and no recurrence in CD99(10D1)-BBz CAR-T cell treated animals on day 59-post amputation, as compared to animals treated with anti-CD19 CAR-T cells (rapid progression of tumors after limb amputation). Furthermore, prolonged xenograft survival was shown after limb amputation in CD99(10D1)-BBz CAR-T cell treated mice as compared to mice treated with CD19 CAR-T cells (FIG. 13A). Additionally, lung metastasis was not evident in mice treated with CD99(10D1)-BBz CAR-T cells, as compared to mice treated with CD19 CAR-T cells, as evident by an increase in the detection of EWS-FLI1 DNA in plasma collected from the treated mice (FIG. 13B).

Taken together, this data shows the CD99(10D1)-BBz CAR-T cell treatment is effective in lysing Ewing sarcoma cells in vitro and reducing tumor burden in vivo and preventing lung metastasis after tumor limb amputation.

### Example 7. Functionality of CD99KO CAR-T Cells Against Ewing Sarcoma

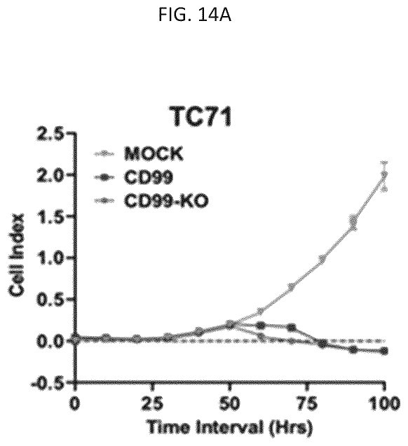

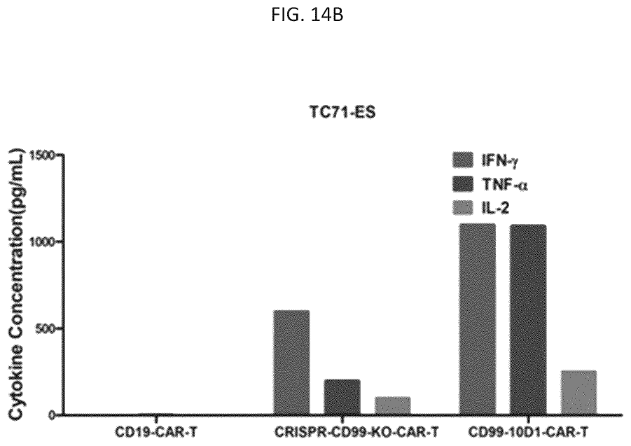

The following experimental example tests the functionality of CRISPR/Cas9 edited CD99KO CD99(10D1)-BBz CAR-T cells against Ewing Sarcoma. Applicants tested the functionality of edited CD99KO CAR-T cells against Ewing Sarcoma cell line, TC71. Ewing Sarcoma cells were treated with unedited CD99(10D1)-BBz CAR-T cells and edited CD99KO CD99(10D1)-BBz CAR-T cells at a 1:1 (tumor cell: CAR-T cell) ratio and changes in growth were measured using XCELLigence real-time measurements that showed complete tumor lysis after treatment with both, unedited CD99(10D1)-BBz CAR-T cells and edited CD99KO CAR-T cells (FIG. 14A). Cytokine production was increased when Ewing Sarcoma cells were treated with unedited CD99(10D1)-BBz CAR-T cells and edited CD99KO CAR-T cells at a 1:1 ratio, as compared to Ewing Sarcoma cells treated with CD19 CAR-T cells (FIG. 14B).

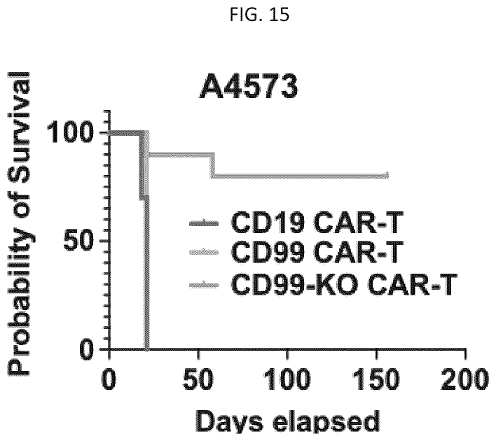

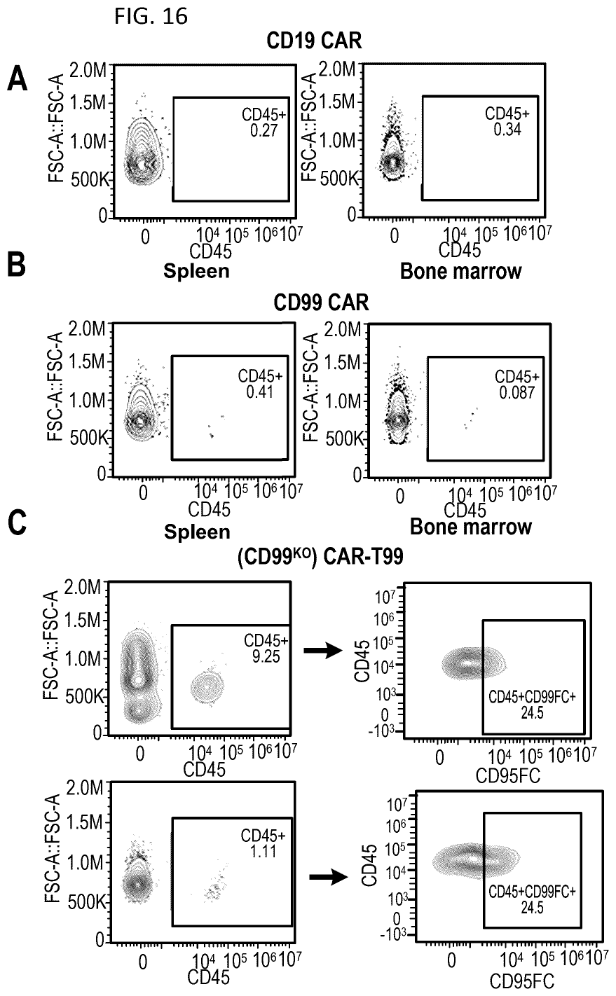

Applicants tested edited CD99KO CD99(10D1)-BBz CAR-T cell treatment in Ewing Sarcoma tumor (A4573-luciferase) cells that were implanted in the mouse pretibial space. After tumor establishment, mice received a single dose CAR-T cell infusion into the tail vein. Applicants found that edited CD99KO CAR-T cell treatment in A4573-Luciferase Ewing Sarcoma mouse model at a single dose of 15×10⁶ cells, lead to complete clearance of tumor burden (monitored by BLI imaging) by day 12 post treatment. Mice treated with unedited CD99(10D1)-BBz CAR-T cells at a dose of 20×10⁶ cells, and mice treated with anti-CD19 CAR-T cells at a dose of 20×10⁶ cells showed no tumor clearance. Furthermore, mice treated with edited CD99KO CD99(10D1)-BBz CAR-T cells showed prolonged xenograft survival of over 150 days post treatment, as compared to treatment with unedited CD99(10D1)-BBz CAR-T cells or CD19 CAR-T cells (FIG. 15). Applicants also found that edited CD99KO CD99(10D1)-BBz CAR-T cells persisted longer in treated mice as compared to unedited CD99(10D1)-BBz CAR-T cells or CD19 CAR-T cells in treated mice (FIG. 16).

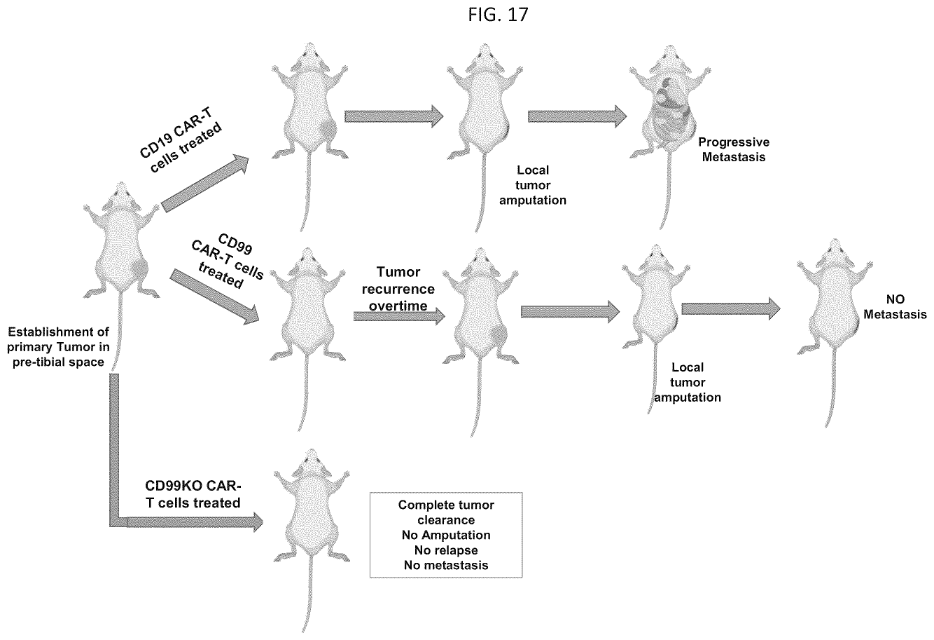

Taken together, this data shows the CRISPR/Cas9 edited CD99KO CD99(10D1)-BBz CAR-T cell treatment is effective in eradicating Ewing Sarcoma cells in vivo with no metastasis and without the necessity of limb amputation (FIG. 17).

1. An anti-CD99 single chain variable fragment (scFv) comprising the amino acid sequence of SEQ ID NO: 12.

2. A chimeric antigen receptor (CAR) polypeptide comprising an anti-CD99 scFv comprising the amino acid sequence of SEQ ID NO: 12.

3. The CAR polypeptide of claim 2, wherein the CAR polypeptide comprises, from N-terminus to C-terminus:
i) a signal peptide;
ii) an anti-CD99 antigen binding domain comprising the anti-CD99 scFv of claim 1;
iii) a hinge domain comprising a CD8 hinge polypeptide;
iv) a transmembrane domain comprising a CD8 transmembrane polypeptide;
v) a costimulatory domain comprising a 4-1BB costimulatory polypeptide; and
vi) an activation domain comprising CD3ζ activation polypeptide.

4. The CAR polypeptide of claim 3, wherein:
the CD8 hinge polypeptide comprises the amino acid sequence of SEQ ID NO: 16;
the CD8 transmembrane polypeptide comprises the amino acid sequence of SEQ ID NO: 20;
the 4-1BB costimulatory polypeptide comprises the amino acid sequence of SEQ ID NO: 24; and
the CD3ζ activation polypeptide comprises the amino acid sequence of SEQ ID NO: 26.

5. The CAR polypeptide of claim 4, wherein the CAR polypeptide comprises the amino acid sequence of SEQ ID NO: 32.

6. The CAR polypeptide of claim 2, wherein the CAR polypeptide comprises, from N-terminus to C-terminus:
i) a signal peptide;
ii) an anti-CD99 antigen binding domain comprising the anti-CD99 scFv of claim 1;
iii) a hinge domain comprising a CD28 hinge polypeptide;
iv) a transmembrane domain comprising a CD28 transmembrane polypeptide;
v) a costimulatory domain comprising a CD28 costimulatory polypeptide; and
vi) an activation domain comprising CD3 activation polypeptide.

7. The CAR polypeptide of claim 6, wherein:
the CD28 hinge polypeptide comprises the amino acid sequence of SEQ ID NO: 14;
the CD28 transmembrane polypeptide comprises the amino acid sequence of SEQ ID NO: 18;
the CD28 costimulatory polypeptide comprises the amino acid sequence of SEQ ID NO: 22; and
the CD3ζ activation polypeptide comprises the amino acid sequence of SEQ ID NO: 26.

8. The CAR polypeptide of claim 7, wherein the CAR polypeptide comprises the amino acid sequence of SEQ ID NO: 34.

9. A nucleic acid molecule comprising at least one nucleic acid sequence encoding an anti-CD99 scFv or a CAR polypeptide, wherein said anti-CD99 scFv comprises the amino acid sequence of SEQ ID NO:12, and wherein said CAR polypeptide comprises said anti-CD99 scFv.

10. (canceled)

11. A cell expressing a CAR polypeptide comprising an anti-CD99 scFv comprising the amino acid sequence of SEQ ID NO: 12.

12. The cell of claim 11, wherein the cell is an immune cell.

13. The cell of claim 12, wherein the immune cell is a T cell, NK cell, NK-like cell, NKT cell, or cytokine induced killer (CIK) cell.

14. The cell of claim 11, wherein the cell does not express CD99.

15. The cell of claim 14, wherein the cell has been genetically modified to not express CD99.

16. A population of cells, wherein said population of cells comprises cells expressing a CAR polypeptide comprising an anti-CD99 scFv comprising the amino acid sequence of SEQ ID NO:12.

17. A method of treating cancer in a subject, the method comprising administering to the subject one or more amounts of a population of cells, wherein said population of cells comprises cells expressing a CAR polypeptide comprising an anti-CD99 scFv comprising the amino acid sequence of SEQ ID NO:12.

18. A method of preventing cancer metastasis in a subject, the method comprising administering to the subject one or more amounts of a population of cells, wherein said population of cells comprises cells expressing a CAR polypeptide comprising an anti-CD99 scFv comprising the amino acid sequence of SEQ ID NO:12.

19. The method of claim 17, the method further comprising administering to the subject at least one additional therapy.

20. The method of claim 19, wherein the at least one additional therapy comprises at least one of radiation therapy, chemotherapy, and surgery.

21. The method of claim 17, wherein the cancer is diffuse intrinsic pontine glioma (DIPG), acute myeloid leukemia (AML) or Ewing sarcoma.
